(19) United States
(12) Patent Application Publication
(10) Pub. No.: US 2024/0299218 A1
(43) Pub. Date: Sep. 12, 2024

(54) SKIN ENVIRONMENT MANAGEMENT SYSTEM WITH VIEWING WINDOW AND DYNAMIC PRESSURE REDUCTION

(71) Applicant: Cardiac Assist Holdings, LLC, Ann Arbor, MI (US)

(72) Inventors: Allen Kantrowitz, Ann Arbor, MI (US); Andrew Ziegler, Ann Arbor, MI (US); Ahmed Mohammed, Ann Arbor, MI (US); Barry N. Gellman, Ann Arbor, MI (US)

(73) Assignee: Cardiac Assist Holdings, LLC, Ann Arbor, MI (US)

(21) Appl. No.: 18/441,080

(22) Filed: Feb. 14, 2024

Related U.S. Application Data
(60) Provisional application No. 63/450,720, filed on Mar. 8, 2023.

(51) Int. Cl. A61F 13/05; A61F 13/00; A61F 13/02; A61M 1/00; A61M 25/02; A61M 39/02

(52) U.S. Cl. CPC A61F 13/05; A61F 13/0266; A61M 1/96; A61M 39/0247; A61F 2013/00412; A61F 2013/00846; A61M 2025/0266; A61M 2039/0288

(57) ABSTRACT

A skin environment management system is provided that controls environmental conditions on a portion of skin of a subject. The system includes a dressing that includes a skin interface, a dressing base attached to the skin interface, and a cover reversibly-openable and connected to the dressing base to together define a volume. The cover sealable to the skin interface, and the dressing is configured to be positioned over the portion of the skin, the dressing defining an environment in the volume surrounding the portion of the skin. The system further includes a pump in fluid communication with the environment by a first tube, the pump configured to apply a reduced pressure vacuum to the environment, and a controller configured to dynamically control operation of the pump. A dressing for use with a skin environment management system is also provided.

### RELATED APPLICATIONS

This application claims priority benefit of U.S. Provisional Application Ser. No. 63/450,720 filed 8 Mar. 2023, the contents of which are hereby incorporated by reference.

### FIELD OF THE INVENTION

The present invention in general relates to medical devices and systems and, in particular to, percutaneous access devices for reducing the likelihood of a clinically significant infection at the site of cutaneous access. More specifically, the invention provides devices for inhibiting microbial ingress proximal to the cutaneous access point with dynamic vacuum draw and the ability to non-invasively inspect and assess the tissues adjacent to the skin entry point.

### BACKGROUND OF THE INVENTION

Chronic kidney disease (CKD) is the 9th leading cause of death in the US affecting approximately 37 million people (15% of the adult population). Annually, over 500,000 patients receive renal replacement therapy in the form of dialysis treatment, for which ˜20% can be placed on peritoneal dialysis (PD) home therapy. It is generally understood that increased utilization of PD will have a significant impact on the safety and effectiveness of care of the US dialysis population.

Entry sites for percutaneous catheters, access devices (PAD), or other skin penetrating implantable medical devices are susceptible to bacterial growth, infection, and problems healing. One reason for this is that such sites are not conducive to skin regeneration and repair processes needed to form an immunoprotective seal against infection about the periphery of the appliance. These problems arise in part because new cell growth and maintenance is typically frustrated by the considerable mechanical forces exerted on the interfacial layer of cells due to the presence of the PAD or other skin penetrating device. Without such immunoprotective seals, skin penetrating devices act as a situs for repeated failure of local skin reparative processes, biofilm formation and infection at the site of insertion or along the surface of the device; the biologic microenvironment adjacent to the device exterior acting as a microbial conduit past the dermal barrier defense complex. It is appreciated that microbial colonization is intended to have a meaning distinct from infection, with infection referring to a host cytotoxicity in which the host immune system no longer able to maintain microbial statis. Infection of the outer surface of specific devices in this class is well documented with catheter hub and catheter-related bloodstream infections being major complications for patients with indwelling catheters (e.g., Safdar and Maki, Intensive Care Med. 2004 January; 30(1):62-7; Saint et al., Infect Control Hosp Epidemiol. 2000 June; 21(6):375-80).

The aforementioned infections are commonly referred to as skin Exit Site Infection (ESI) and are common complication associated with long-term medical treatment modalities that require a long-term indwelling medical appliance device to penetrate the skin, illustratively including peritoneal dialysis, long-term vascular access, drivelines associated with mechanical cardiac assistance, Steinman pins and K-wires. ESI have been associated with (1) cellulitis of subcutaneous tissues; (2) erosion of tissues adjacent to skin exit site; infection along the catheter to deeper planes; and (3) intractable infection and systemic sepsis. ESI is a leading cause of unplanned hospitalizations for peritoneal dialysis (PD) patients. The rate of ESI is 1 episode per 62.6 patient-months or 0.19 episodes per patient-year for PD patients, and adds significant cost due to adverse events, hospitalizations and treatments. FIGS. 1A-3 show various skin entry sit infections.

In order to facilitate repair of the dermal barrier defense complex about the exterior of a PAD to avoid ESI, subject cells are often harvested and grown in culture onto PAD surfaces for several days prior to implantation in order to allow an interfacial cell layer to colonize PAD surfaces in advance of implantation. Unfortunately, cell culturing has met with limited clinical acceptance owing to the need for a cell harvesting surgical procedure preceding the implantation procedure. Additionally, maintaining tissue culture integrity is also a complex and time-consuming task.

As an alternative to cell culturing on a percutaneous access device, vacuum assisted treatment about a percutaneous access device has been attempted with and without the addition of a fibroblast adhesion feature, for example one or more circumferential cuffs made of DACRON®-based random-felt mesh used to promote fibroblast attachment between the outer surface of the catheter and the walls of the surgically created tunnel through the subcutaneous tissues in the vicinity of the catheter exit site. Such “Dacron cuffed” catheters are frequently used by clinicians despite the uncontrolled pore sizes of the Dacron felt which are recognized to include bacterial growth pockets large enough to harbor bacterial colonies but small enough to mechanically frustrate local host cell-based defense mechanisms and promote ESI.

Negative Pressure Wound Therapy (NPWT) systems improve wound healing by removing wound exudates and actively promoting tissue granulation. By providing intermittent or continuous therapy through sub-atmospheric pressure, NPWT technology assists in faster healing of open wounds by several distinct mechanisms, including but not limited to optimization of blood flow in the wound bed; decreasing of local swelling; removal of excess wound exudative fluid that would otherwise support bacterial colonization, and mechanical stabilization of the tissues in the vicinity of the wound.

One intention of NPWT is to remove wound exudate, thereby reducing the combined host cellular debris, reactive wound exudate and debris burden created by microbiologic colonization, collectively termed Bioburden, within the wound site. This decreases localized edema and increases blood flow, which in turn decreases tissue bacterial levels. Additionally, the application of sub-atmospheric pressure produces mechanical deformation or stress within the tissue resulting in fibroblast proliferation, migration and biosynthetic activation with resultant protein and matrix molecule synthesis, enhanced angiogenesis as well as mechanical stabilization and controlled intermittent wound compression and enhanced granulation tissue formation.

By virtue of the technology, NPWT allows clinicians to limit the amount of bioburden accumulating within the wound tunnel microenvironment, thus, protecting the skin and enhancing patient comfort. Furthering the goal of improving patient comfort is the reduction in the number of dressing changes required as well as the reduction in a risk of infection to an exposed wound. NPWT systems typically include disposable tubing, foam wound dressing, adhesive film that covers and seals the wound as well as a controllable pump.

NPWT technology is primarily used for acute or chronic wounds, as well as burns. NPWT technology regulates pressure at the wound site, providing safe and accurate delivery of the prescribed settings through the pump to foster rapid wound granulation, epithelialization, and wound contraction. NPWT are for use across a breadth of medical markets including Acute Care, Long Term Care, Homecare, and Wound Care environments. Of concern, NPWT devices, when used improperly or in certain inappropriate clinical scenarios can lead to a counter-therapeutic environment in the wound vicinity of the healing wound.

FIG. 4 depicts a prior art device generally at 100, as shown in U.S. Pat. No. 10,258,784B2. A cap 102 serves to keep debris from entering the device 100. Preferably, the cap 102 is remote from the surface of the epidermis E. The medical appliance 34 depicted as a catheter and vacuum or hydrodynamic draw tubing 104 pass through complementary openings 106 and 108, respectively formed in the cap 102. The tubing 104 provides fluid communication between a vacuum or hydrodynamic draw source 22 and an inner sleeve 12d. The inner sleeve 12d is characterized by a large and rigid pore matrix 18 in fluid communication to a vacuum source 22 such that the source 22 draws (arrow 22D) tissue fluid and fibroblasts 21 into the sleeve 12d. Sleeve 12d has a surface 24 that is optionally nanotextured to promote fibroblast adhesion. The surface 24 is optionally decorated with a pattern of contoured cell-conveying channels. It is appreciated that inner sleeve 12d optionally includes matrix 26 thereover, a coating substance 27, or a combination thereof. The coating 27 is appreciated to need not cover the entire surface 24. The tissue contacting surface 29 of substance 27 is optionally nanotextured. A flange 112 is provided to stabilize the implanted device 100 within the subcutaneous layer S. A flange 112 is constructed from materials and formed by methods conventional to the art. For example, those detailed in U.S. Pat. Nos. 4,634,422; 4,668,222; 5,059,186; 5,120,313; 5,250,025; 5,814,058; 5,997,524; and 6,503,228.

However, existing devices place a burden on nursing staff and caregivers associated with monitoring the entry site, an associated vacuum reservoir, and the healing status thereof and changing out and any bandages to promote proper healing of a wound. Not only do such monitoring and care requirements create a nursing burden, but such care requirements may also counterproductive to wound healing in that the entry site is not readily visible and each instance of removing the bandage or vacuum reservoir for visual inspection subjects the wound to mechanical forces and microbes that are detrimental to healing the percutaneous access point wound under the dressing.

Thus, there is a continuing need for improved devices for protecting percutaneous access device entry sites from infection, particularly for devices that are equipped with improved environmental controls, pressure controls, and feedback to improve monitoring capabilities as well as encourage and maintain an infection inhibitive seal around percutaneous access device. There is a further need for an effective wound management system to mitigate infection and accelerate healing of the PD catheter skin exit site to increase safety, improve quality of life, reduce healthcare costs, and lessen the burden of wound care on attendant staff.

### SUMMARY OF THE INVENTION

A skin environment management system is provided that controls environmental conditions on a portion of skin of a subject. The system includes a dressing that includes a skin interface, a dressing base attached to the skin interface, and a cover reversibly-openable and connected to the dressing base to together define a volume. The cover sealable to the skin interface, and the dressing is configured to be positioned over the portion of the skin, the dressing defining an environment in the volume surrounding the portion of the skin. The system further includes a pump in fluid communication with the environment by a first tube, the pump configured to apply a reduced pressure vacuum to the environment, and a controller configured to dynamically control operation of the pump.

A dressing for use with a skin environment management system is provided. The dressing includes a pouch formed of a first sheet and a second sheet layered together and sealed along an outer edge to define a chamber therein. A skin barrier is attached to the first sheet at a through hole in the first sheet, as well as a sealable outlet configured to fluidly connect the chamber of the pouch with an environment external to the chamber of the pouch.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIGS. 1A and 1B are photos of a prior art driveline infection at a skin exit site;

FIG. 2 is a photo of a prior art exit site infection illustrating the catheter, contaminated exit site, and subcutaneous location of subcutaneous cellulitis;

FIG. 3 illustrates the prior art four phases for marsupialization of walls of a tunnel: Phase (P)1: beginning of downward epidermal migration; P2 along the subcutaneous tissue, P3 down to the cuff, and P4 the deeper portion of the catheter;

FIG. 4 is a prior art, partial cutaway view of a flanged percutaneous access device (PAD) with relative dimensions of aspect exaggerated for visual clarity;

FIG. 5A illustrates a reduced pressure dressing in a low vacuum state, by way of example in the range of 0 mm Hg to approximately −50 mm Hg relative to atmospheric pressure in accordance with an embodiment of the invention;

FIG. 5B illustrates a reduced pressure dressing in a high vacuum state, by way of example in the range of −50 mm Hg to approximately −125 mm Hg relative to atmospheric pressure in accordance with an embodiment of the invention;

FIG. 6 shows a perspective view of a skin environment management system according to certain embodiments of the present invention;

FIG. 7 shows a side, elevated perspective view of the dressing and tubing of a skin environment management system according to certain embodiments of the present invention;

FIG. 8 shows a perspective view of a controller of the skin environment management system according to certain embodiments of the present invention;

FIG. 9 shows a front, elevated perspective view of a dressing in a partially open position and tubing of the skin environment management system according to certain embodiments of the present invention installed around a catheter;

FIG. 10 shows a first rear, elevated perspective view of a dressing in a fully open position according to certain embodiments of the present invention installed around a catheter;

FIG. 11 shows a second rear, elevated, perspective view of a dressing in a partially open position with tubing now shown with the skin environment management system according to certain embodiments of the present invention installed around a catheter;

FIG. 12 shows a front, elevated perspective view of a dressing in a closed position and tubing of the skin environment management system according to certain embodiments of the present invention installed around a catheter and showing the transparent window therein;

FIG. 13 shows a rear, elevated perspective view of a dressing in a closed position and tubing of the skin environment management system according to certain embodiments of the present invention installed around a catheter;

FIG. 14 is a schematic drawing of the skin environment management system according to certain embodiments of the present invention;

FIG. 15 is a schematic diagram illustrating an overall view of communication devices, computing devices, and mediums for implementing embodiments of the invention;

FIG. 16 is a side view of a bandage portion of the skin environment management system according to certain embodiments of the present invention installed around a catheter;

FIG. 17 is a first exploded view of a controller of an inventive system;

FIG. 18 is a second exploded view of the controller depicted in FIG. 17;

FIG. 19A shows a dressing according to embodiments of the present invention having a zipper sealable access opening;

FIG. 19B shows the dressing of FIG. 19A adhered to the skin of a patient and in an open position;

FIG. 19C show the dressing of FIGS. 19A and 19B adhered to the skin of a patient and in a closed position;

FIG. 20A shows a dressing according to embodiments of the present invention having a dome-shaped cap sealable access opening;

FIG. 20B shows the dressing of FIG. 20A adhered to the skin of a patient and in an open position;

FIG. 20C show the dressing of FIGS. 20A and 20B adhered to the skin of a patient and in a closed position;

FIG. 21A shows a dressing according to embodiments of the present invention having a pH buffering skin barrier;

FIG. 21B shows the dressing of FIG. 21A adhered to the skin of a patient;

FIG. 21C show a graph comparing the buffering action of a conventional skin barrier and a skin barrier as used in FIGS. 21A and 21B;

FIG. 22A shows an exploded view of a three part stepped-cone connection fixture according to embodiments of the present invention;

FIG. 22B shows the connection fixture of FIG. 22A installed on a dressing according to embodiments of the present invention;

FIG. 23A shows a box containing several soft wafer stick skin barriers prior to their use; and

FIG. 23B show several soft wafer stick skin barrier applied around a wound site of a patient.

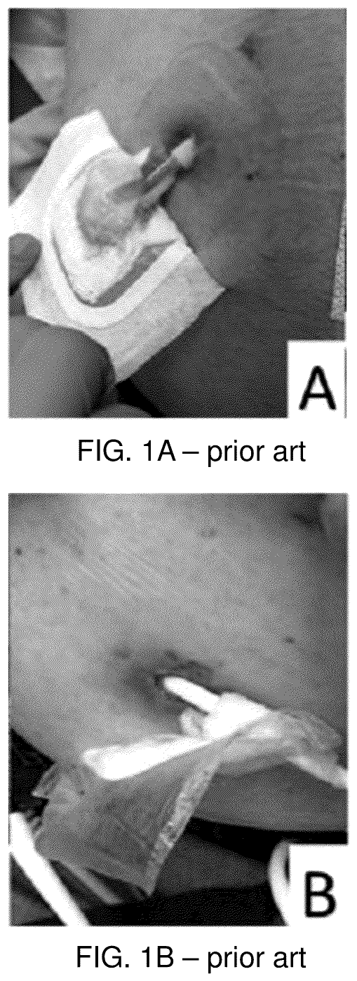

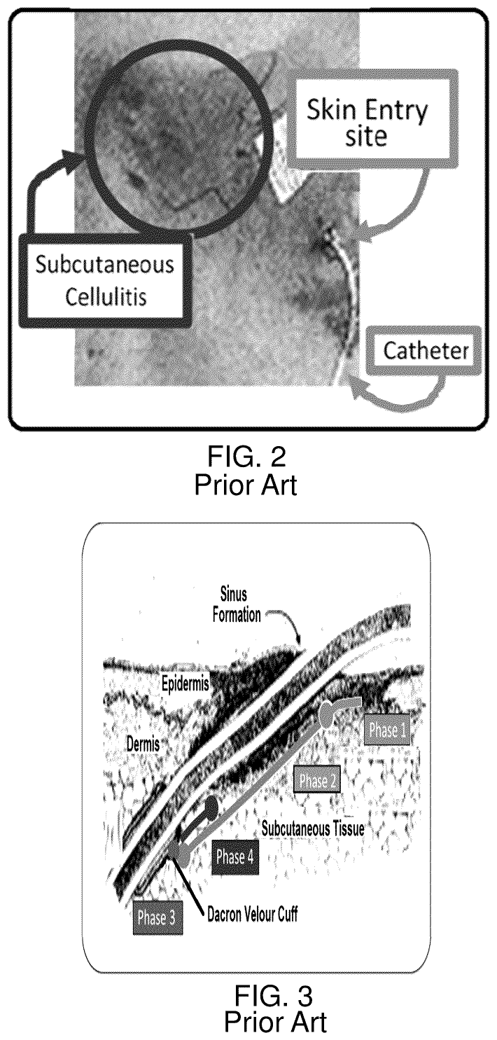

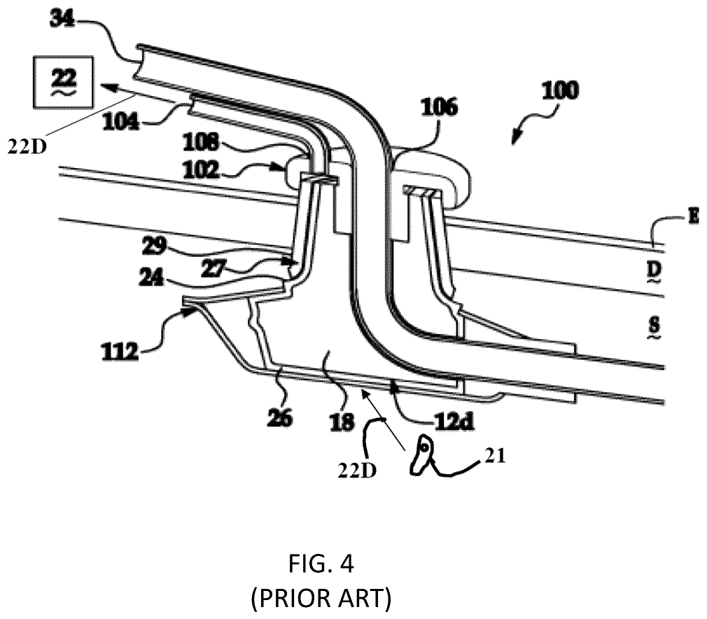

### DETAILED DESCRIPTION OF THE INVENTION

Embodiments of the present invention have utility in controlling the environment in a portion of subject skin and viewing same without compromise of the environment. In particular, when the portion of subject skin includes a medical appliance skin exit site healing and stabilization thereof are promoted. An inventive system is equipped with environmental controls, pressure controls, and feedback to promote healing around an exit site. Embodiments of the present invention have further utility as effective skin environment management systems to mitigate infection and accelerate healing of skin exit site to increase safety, improve quality of life, reduce healthcare costs, and lessen the burden of compromised skin on attendant staff, such as nursing staff and caregivers. By way of example, Peritoneal Dialysis Therapy is facilitated with optimized healing associated with the present invention.

Numerical ranges cited herein are intended to recite not only the end values of such ranges, but the individual values encompassed within the range and varying in single units of the last significant figure. By way of example, a range of from 0.1 to 1.0 in arbitrary units according to the present invention also encompasses 0.2, 0.3, 0.4, 0.5, 0.6, 0.7, 0.8, and 0.9; each independently as lower and upper bounding values for the range.

The following description of various embodiments of the invention is not intended to limit the invention to these specific embodiments, but rather to enable any person skilled in the art to make and use this invention through exemplary aspects thereof.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

It is noted that previous efforts have concentrated on removing moisture or humidity from wound areas, however it is observed in the present invention that a level of moisture is required to allow fibroblasts to actively attach to an inserted device and to promote the establishment of intact biological barrier function of the stratum corneum layer of skin and for wound healing in general. It is also noted that moisture and pressure levels may be needed to change as the wound healing process progresses through different stages. In some inventive embodiments, a controller provides dynamic control of pressure, humidity, or both. It is further noted that pressure levels may require dynamic changes to preclude skin prolapse around an implanted device or otherwise damage to the capillary bed underlying the dressing 200.

In some inventive embodiments, a physiological sensor samples the volume within the dressing 200 and prior to the reservoir. It has been surprisingly discovered that the volume of the dressing owing to a lack of atmospheric turnover affords low detection limits of sensed conditions and therefore early signaling to an attendant of infection or other deleterious condition. A suitable sensor is detailed in A. Pusta et al. “Sensors for the Detection of Biomarkers for Wound Infection,” Biosensors: 2022; 12(1):1; as well as in U.S. Pat. No. 10,791,984 B2. The sensor includes a processor and memory. The processor is in electrical communication with the memory and in some inventive embodiments includes a program memory and data memory. The program memory includes processor-executable program instructions implementing encoded processor-executable program instructions configured to implement an optional OS (Operating System) or Application Software. Preferably, the processor is wirelessly communicatively and operably coupled with the I/O (Input/Output) interface while operating artificial intelligence dressing volume detection. It is appreciated that neural network, machine learning, artificial intelligence, or digital signal processing functions are readily completed remotely relative to the dressing controller with resort to a host server. By coupling two or more such sensors from disparate users, all the communicating sensors become more adept at detecting infection, skin prolapse, and other deleterious conditions associated with inventive dressing operation. The resulting trained AI model can be licensed for financial remuneration. The I/O interface thus includes a wireless network interface, as a Wi-Fi or BLUETOOTH® interface. The network interface may be a Bluetooth® interface. The processor in some inventive embodiments is communicatively and operably coupled with the multimedia interface as a way for an attendant to monitor the dressing condition, with the multimedia interface having interfaces illustratively adapted to input and output of audio, video, and image data. The multimedia interface in some inventive embodiments may include one or more still image camera or video camera. Illustrative of such multimedia interfaces are personal computers, servers, tablet PCs, smartphones, or other computing devices. The operably linked sensors can form a computer network in a manner as to distribute and share one or more resources, such as clustered computing devices and server banks/farms. Various arrangements of such general-purpose multi-unit computer networks suitable for implementations of the disclosure, their typical configuration, and standardized communication links are well known to one skilled in the art. processing, such as, for example, neural networks, machine learning, artificial intelligence, image recognition, or digital signal processing.

It is appreciated that by positioning such a sensor in the controller of an inventive device, the skin-contacting dressing complexity is reduced, consistent with the disposable nature thereof.

While the present invention is mostly commonly deployed with a human subject receiving a medical appliance that penetrates through the patient skin, it is appreciated that the present invention is equally suitable for veterinary subjects that include non-human primates, cows, horses, sheep, rodents, dogs, cats, avians, and reptiles. Furthermore, while the portion of subject skin to be encompassed is contemplated to be medical appliance that penetrates through the patient skin, it is appreciated that any skin condition that benefits from a controlled environment as so provided and observed benefits from the present invention. These other conditions illustratively include an ulcerative wound, a skin infection, a surgical incision closed in a water-tight fashion by “primary intention” (edge-to-edge water-tight skin apposition across the incision by use of any skin closure method including non-absorbable skin sutures, absorbable skin suture, absorbable staples, non-absorbable staples, and the like), a surgical incision closed in a loose fashion by “primary intention” (edge-to-edge loose skin apposition allowing egress of reactive wound fluid from deeper layers of the wound across the dermal/epidermal layers by use any skin closure method including non-absorbable skin sutures, absorbable skin suture, absorbable staples, non-absorbable staples, and the like), a surgical incision closed in a loose fashion by “primary intention” with one or more surgical drains [e.g. Penrose drain, etc. . . . ] included within the suture “loose primary repair line” (edge-to-edge loose skin apposition with included surgical drains allowing egress of reactive wound fluid from deeper layers of the wound across the dermal/epidermal layers), or any combination of these surgical wound closure techniques.

Embodiments of the inventive skin environment management system (SEMS) 199 are designed to improve healing while minimizing the risk of infection by enhanced monitoring using sensors, providing for visual inspection without a need to remove the dressing, and providing moisture and temperature controls. According to embodiments, the SEMS minimizes risk of exit site infection by several mechanisms including the reduction of bioburden in the skin exit environment in the acute, subacute, and chronic phases of the PD catheter post-implant. An inventive SEMS enables visualization of the portion of skin subject thereto without tampering with the inventive dressing by providing the following optional features. A window to visualize the underlying skin area adjacent to the exit-site for signs of infection and/or the presence of exudate as well as a reversibly-openable hatch, lid or covering which permits direct clinical inspection and clinical manipulation of the wound. These observations and clinical manipulations assist attendant care in determining whether to change the dressing or if the environment should be treated in some form such as being flushed with a therapeutic fluid, gas, medication, or any other change in medical care that may even include removal of the device change in antibiotics, debridement, or combination thereof. According to some inventive embodiments, strain relief for a catheter is present to limit transmission of repetitive trauma of the catheter-skin relationships due to movement, tension, and twisting of the catheter. The ability of the combined dressing/controller system to measure and monitor pressure, mechanical strain, humidity, temperature, volatile or soluble chemicals emitted by the underlying skin area environment, skin microvascular activity or a combination thereof at the underlying skin area or appliance exit site is thus advantageous. By way of example, a closed-loop vacuum control feedback circuit is used to maintain a constant humidity level, which is for example maintained at 92% relative humidity. A controlled localized vacuum (ranging between −125 mm Hg and zero) at the underlying skin area adjacent to exit-site, while avoiding subjecting the neighboring normal epidermis to unnecessary levels of vacuum and the attendant risk of hypoperfusion-dependent or mechanical-based epidermal/dermal injury. Intermittent cycling of the vacuum to serves to aid mobilization of bioburden from the underlying exit tunnel and the margin of the exit site, and serves, as well, to optimize tissue approximation to the outer surface of the medical implant and also to mechanically stabilize the medical device relative to the skin. Additionally, the periodic cycling of vacuum can be algorithmically modulated to optimize skin microvascular activity in the skin subjacent to the dressing. An intentionally controlled leak allows for air exchange within the dressing within embodiments of the inventive SEMS and creates the possibility of rapid detection of volatile compounds which had been emitted into the local atmosphere adjacent to the wound within the dressing system. A strain gauge and/or a tissue-perfusion sensor are optionally provided in some inventive embodiments to permit the monitoring of compressive forces on the skin adjacent to a medical appliance exiting the skin under the dressing 200. As noted above, it is appreciated that a strain gauge as a subset of sensors used to monitor the dressing volume is readily interfaced to other devices to create a neural network, machine learning, or artificial intelligence information sharing as to conditions thereby allowing for improved interventions to mitigate complications. A fluid collection canister 400, which according to some inventive embodiments is 35 ml, is provided to hold biohazard fluid to be discarded when full.

Embodiments of the inventive skin environment management system combine the use of vacuum assist technology with a novel dressing to control the environment within and around the underlying skin area or medical device penetration/exit site. Embodiments of the inventive SEMS employ algorithms to control local atmospheric parameters within the dressing adjacent to the wound, such as but not limited to pressure, humidity, and gas composition by applying appropriate vacuum levels and dynamically modifying the atmospheric parameters in response to certain threshold values being communicated by the sensors or other programmed inputs. The local intra-dressings atmospheric control algorithms, combined with an algorithmically controllable leak or active venting approaches, allow air exchange in the dressing, and represent a new innovative approach to underlying skin care or implanted appliance installation. Unlike other negative pressure dressings, embodiments of the inventive SEMS are specifically designed to treat the local conditions unique to the skin exit site of a medical appliance penetration through the skin of a subject. Such embodiments of the inventive device are designed to stabilize passage of a medical appliance through a dressing with a strain relieving component to protect the exit site from trauma while applying negative pressure to treat the skin portion in a highly controlled manner. Of importance, the design of the inventive dressing anticipates and limits the mechanical forces employed during both the attachment processes and the detachment processes between skin, transiting medical device, and dressing. In clarification of this inventive point: Prior art dressings have been optimized for ease of attachment and frequently will achieve a vacuum seal between dressing and transiting medical appliance by means of liberal application of one or more layers of thin adhesive films to bridge the gaps between dressing and outer surface of the transiting medical device. Vacuum seals created by such prior art means are awkward and difficult to subsequently detach without subjecting the interface between the transiting medical device and the skin to large counter-therapeutic mechanical forces frequently resulting in injury to the tissues adjacent to the transiting medical device. Embodiments of the inventive SEMS provide a fully vacuum sealed system (via injection molded, adhesives, thermoformed, extruded and/or insert molded components) to create the desired hermetic seal which, by inventive design, allow for vacuum sealing and unsealing to occur between dressing and the outer surface of the transiting medical device while avoiding subjugation of the interface between the transiting medical device and the skin to large counter-therapeutic mechanical forces thereby minimizing injury to the tissues adjacent to the transiting medical device.

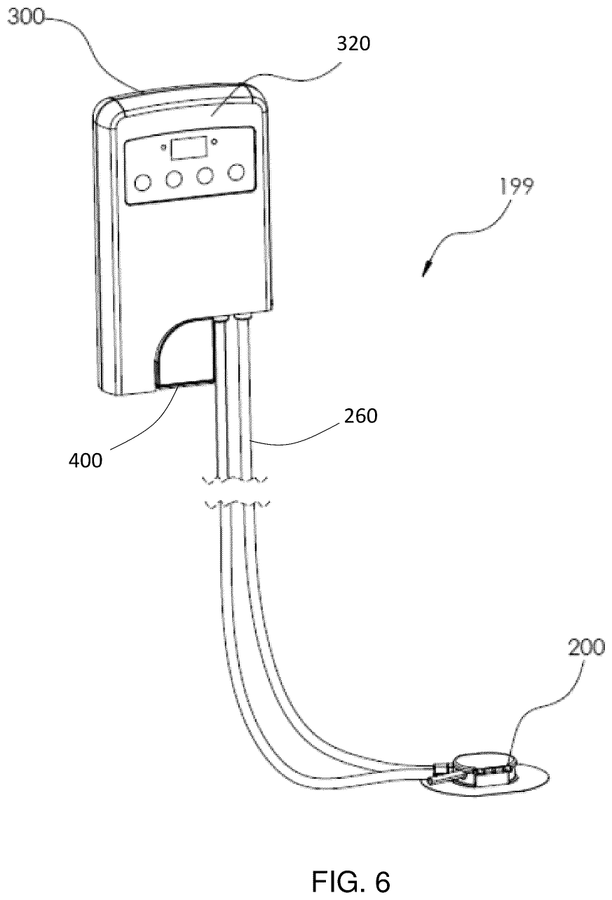

As shown in FIG. 6, embodiments of the present invention provide a skin environment management system (SEMS) 199 with vacuum assist therapy (VAT) to provide healing therapy to a portion of subject skin encompassed. While the inventive system is depicted in the accompanying figures with a generic embedded catheter, it is appreciated that it is applicable to a variety of such appliances including a percutaneous access device (PAD), a Steinman pin, and a Kirschner wire, a peritoneal dialysis (PD) catheter, and a chronic indwelling vascular access catheter that requires skin penetration. The system 199 includes of a controller 300 with a vacuum pump 401 and a fluid collection canister 400 and a specially designed dressing 200 that may be unique for each different application to which the invention is supplied. Tubing 260 interconnect the dressing 200 to the pump 401. According to some inventive embodiments, the tubing 260 includes a clip 268 configured to releasably connect to a corresponding clip 310 provided on the controller 300. The clips 268, 310 may be provided in a single size so as to fit together no matter the size of the tubing 260 used in the system 199. Thus, the controller 300 is designed to work with a family of implantable medical devices and tubing 260. Similarly, the dressing 200 is available in a variety of sizes to enable use with implantable medical devices of various sizes and to ensure a best fit with the anatomy of a subject. According to embodiments, the inventive SEMS 199 additionally includes atmospheric and/or physiologic sensors such as humidity, pressure, and/or temperature sensors 240 integrated into the dressing 200 to provide physiologic parameters for time-varying control of the milieu surrounding the catheter exit site, an exudate reservoir, and a strain relieving fixture and/or alert patient and/or caregivers to changes in the clinical status of the underlying skin area which would impact further care rendered to the patient. According to embodiments, all of the components of the inventive SEMS 199 are single use sterile components that are configured to be disposed after use. The system's placement helps optimize subject care, reduces exit site infections, and reduces burdens on attendant staff associated with care requirements. According to inventive embodiments, the skin environment management system promotes the formation of a natural biologic seal between the skin and the medical appliance to form a barrier to microbial invasion into the body that accelerates healing and mitigates ESI. According to embodiments, the present invention is configured to provide a reduced pressure dynamic draw vacuum to the volume including a portion of subject skin including a device exit site 24 hours a day, 7-days a week within minimal intervention from attendant staff, who may observe the wound condition through the dressing window.

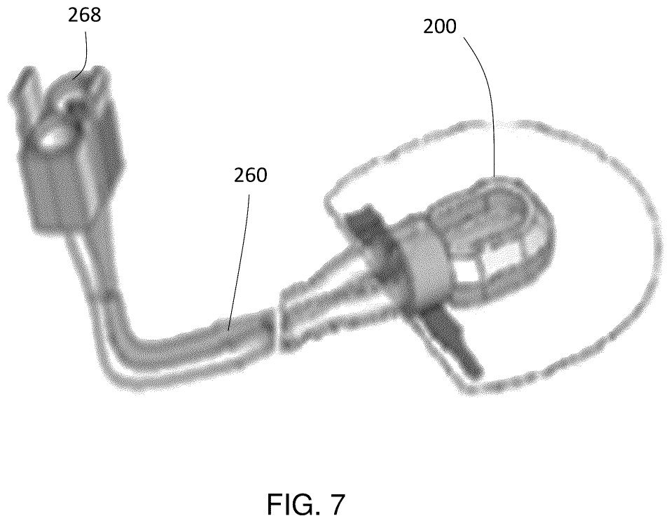

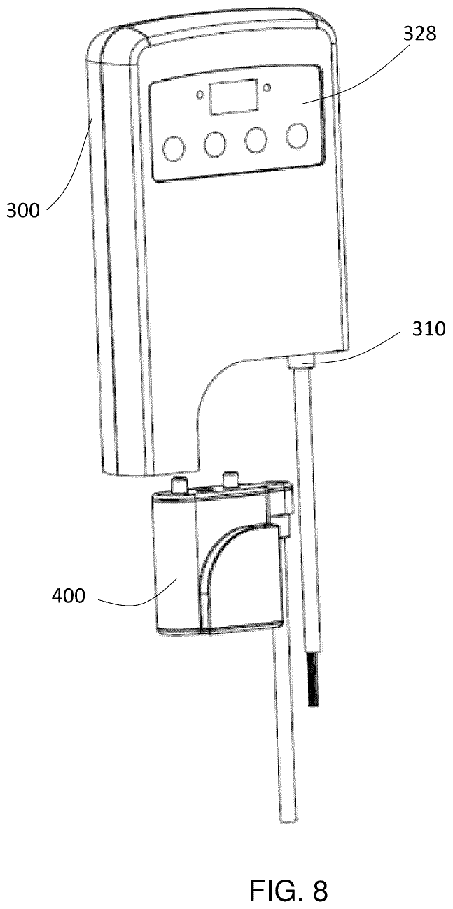

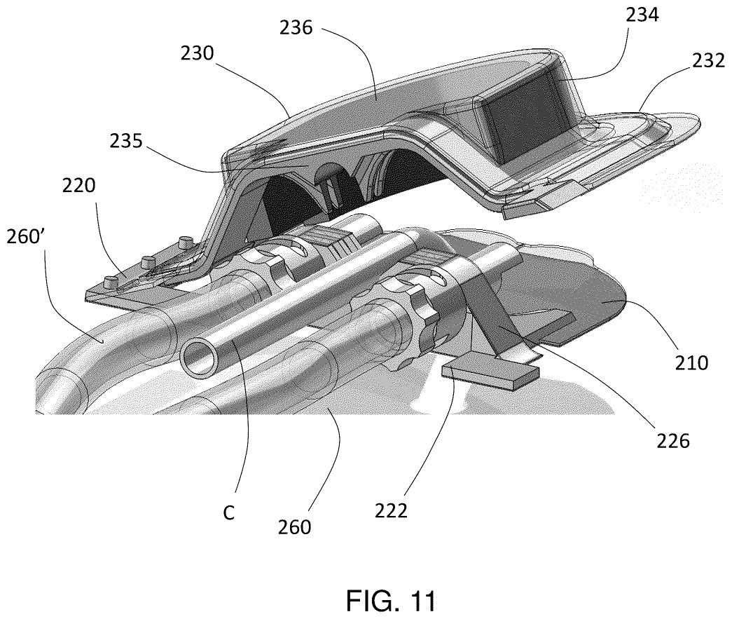

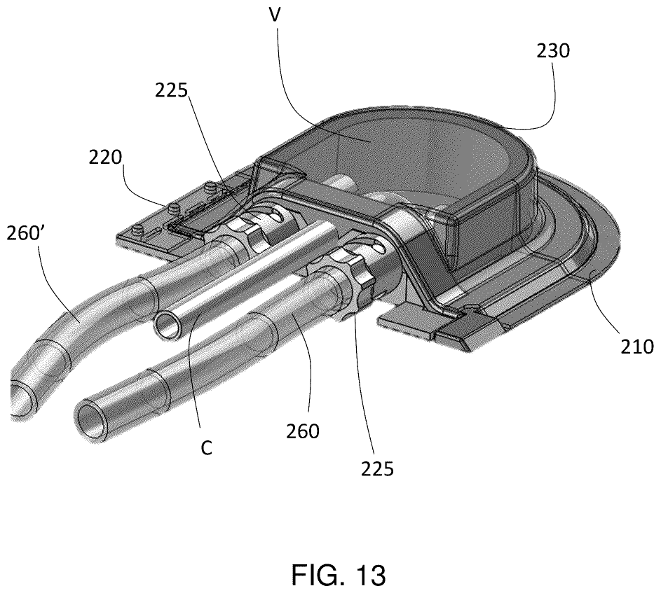

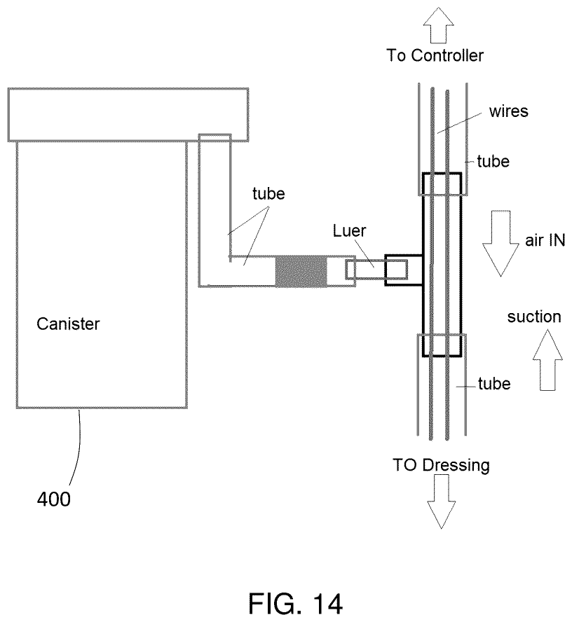

According to some inventive embodiments as shown in FIGS. 7-14, in which like reference numerals have the same meaning ascribed thereto in reference to FIG. 6, the dressing 200 is a negative pressure dressing that is configured to be applied to the skin of a subject around an exit site of a skin penetrating appliance, such as a catheter C. The dressing is configured to maintain a negative pressure applied to the skin at the site where the skin penetrating device emerges through the epidermis. Furthermore, the dressing 200 is configured to provide stability to the skin penetrating device, such as a catheter C, and also provide visibility of the volume including the skin portion without the need to remove the dressing from the skin of the subject. The dressing 200 may be applied and managed by a medical professional or trained personnel. The inventive dressing 200 is also configured to maintain a negative pressure seal around the exit site of a medical appliance and provide access to draw and collect exudate from the volume including the portion of subject skin. According to embodiments, the applied vacuum is not to exceed −125 mm Hg and in still other embodiments is between −1 and −120 mm Hg. According to some inventive embodiments, the intended duration of use for the inventive SEMS 199 is seven days continuously. By stabilizing the skin penetrating medical appliance, such as a catheter C, for seven days post-placement, the surrounding tissue is permitted to heal and engage the skin penetrating device to stabilize it. Without intending to be bound to a particular theory, fibroblast infiltration around the implant site is an important step in stabilization and creating of a rejuvenated skin barrier around the exit site. Otherwise, movement of the skin penetrating medical appliance relative to the skin in the vicinity of the skin exit site of the appliance may result in a local trauma to tissues adjacent to the skin exit site of the appliance resulting in products of tissue damage adding to local bioburden and local nidus for harboring and supporting local bacterial growth. Accordingly, embodiments of the present invention provide a strain relief integrated into the dressing 200 to provide support against counter-therapeutic relative movement between the medical appliance and tissues adjacent to the skin exit site of the medical appliance. An skin-adherent adhesive construct that mitigates against damage to the appliance when a dressing is removed or exchanges is known to the art as detailed in US 2021/0161720A1.

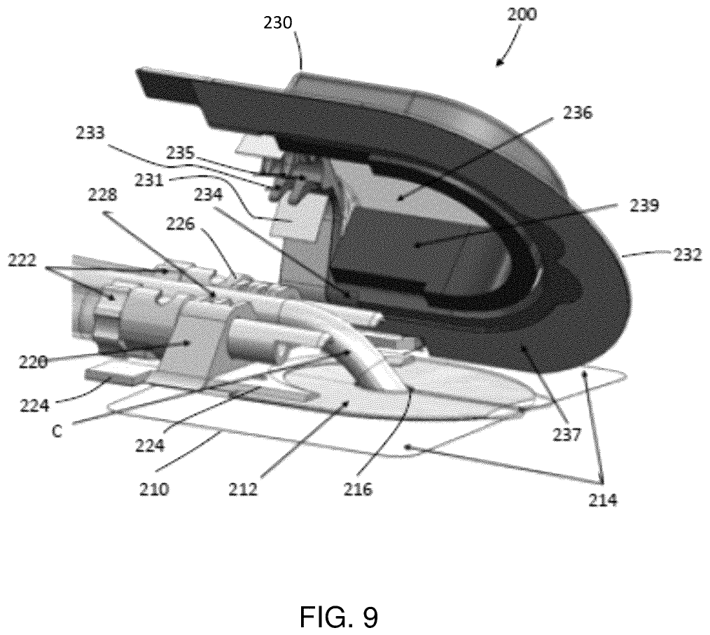

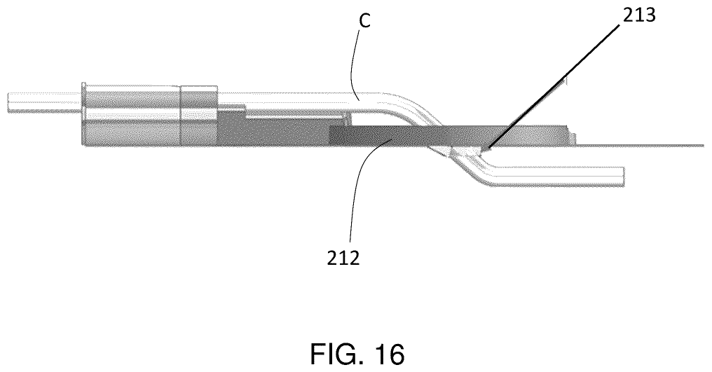

As shown in greater detail in FIGS. 9-13, in which like reference numerals have the same meaning ascribed thereto with respect to previously detailed drawings, embodiments of the dressing 200 include a skin interface 210 that is configured to seal to the skin of a subject. The skin interface 210 includes a bandage portion 212 that is configured to surround a portion of skin of the subject, which may include a medical appliance exit site, and a release liner portion 214 that is configured to cover the bandage portion 212 and seal to the skin of the subject. According to some inventive embodiments, the bandage portion 212 does not include any adhesive so as to avoid subjecting the skin to deleterious forces when the bandage portion 212 is removed and possibly replaced. According to other inventive embodiments, the bandage portion 212 is a gel material dressing. Gel material suitable for a dressing herein illustrative include chlorhexidine (CHX) gel material (an active antimicrobial agent-supplied by PREVAHEX®). The gel material can be fabricated with a pre-made center hole to receive the catheter, C. The gel material, regardless of type, is applied about the emerging catheter, C and advanced to the skin to create a skin barrier. This gel has four potential functions herein that may include: a) as an antimicrobial agent to reduce infection; b) to absorb moisture from the wound; c) to protect the dermis/epidermis from bariatric injury due to direct vacuum exposure; d) is translucent/clear to visualize the puncture site. As the gel absorbs moisture it swells. In some embodiments, the atmospheric exchange inside the dressing chamber, created by the intentional controlled vent and/or non-calibrated air leaks into the dressing chamber, allows for transfer of moisture from the hydrated gel to the exhaust system of the pump controller. According to some inventive embodiments, as shown in FIG. 16, the bandage portion 212 is circular disc (or any other shape) of skin dressing with a unique hole structure in the center, creating only a limited margin ring about the catheter puncture site that is exposed to the negative pressure when a catheter dressing is applied. One means of accomplishing this is with radial slits, for example 3 to 6 slits, provided about the puncture site. The radial tangs between adjacent slits are herein referred to as “petals” 213. That portion of the catheter adhesive dressing defined by the combined area of the petals may be “adhesive-free”. The bandage portion 212 is supplied with both and lower release liner 214 and an upper release liner (opposing side duplicate of 214—not shown). The bandage portion 212 is slit and liners are spread open to receive the catheter diameter. The petals 213 contain no adhesive and permit easy sliding along the shaft of the catheter C. The bandage portion 212 is then advanced to the skin and the lower release liner removed and adhered to the skin. Should the petals 213 face outward, the catheter C may be advanced slightly into the body to reverse the petal direction. Adhesive bandages suitable for long term skin contact are well-known to the art and include aspects such as microvents, gel compositions and the like and are detailed in exemplary form in US 2020/0397930; U.S. Pat. Nos. 6,592,889; and 6,180,544. When properly placed, the petals 213 will bend from the plane of the epidermis to follow the outer wall of the catheter. The portions of the petals remaining undisturbed in the plane of the epidermis will remain in immediate adjacency. However, the portions of the petals which have bent to follow the outer wall of the catheter will, by simple geometric principles, spread apart from their adjacent neighboring petals. This spreading will allow fluid communication of the vacuum to portions of the wall of the subcutaneous tunnel. The pattern will allow precise accommodation of the Catheter Dressing to variations in the outer diameter of the catheter.

The release liner 214, however, includes an adhesive on a skin side thereof. The adhesive of the release liner 214 is configured to stick and seal to the skin of a subject and to the bandage portion 212. The release liner 214 is a full skin dressing to protect the skin that would otherwise be subjected to a vacuum exposure, given that skin that is subjected to long-term vacuum exposure is susceptible to bariatric injury, tissue blistering and/or de-epithelialization. The side of the release liner 214 opposite to the side on which the adhesive is provided is a smooth surface. It is appreciated that the release liner 214 may have laser drilled holes therein to provide for skin perspiration venting. According to embodiments, the skin interface 210 is formed from a compliant, flexible material that easily conforms to any contours of the skin and rest of the dressing 200. An opening 216 is provided through the bandage portion 212 and the release liner portion 214. The opening 216 is configured to allow a catheter C or other skin penetrating medical appliance or device to pass through the bandage 212 and the release liner 214 of the dressing 200. According to embodiments, the opening 216 in the skin interface 210 is a perforation that is configured to be opened by a user. According to embodiments, the opening 216 in the skin interface 210 is sized to correspond to a diameter of the catheter C. The size of the skin interface 210 corresponds to the size of a cover 230 of the dressing 200, which is described in further detail below.

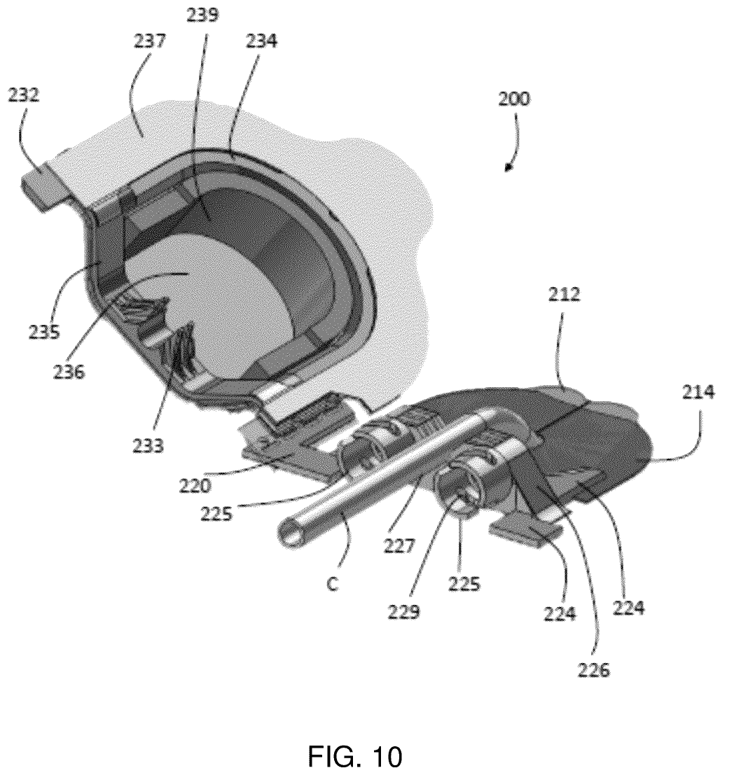

The dressing base 220 is attached to the skin interface 210, particularly to the release liner 214 of the skin interface 210. The dressing base 220 is configured to provide support to the catheter C and to provide attachment points 222 for tubing 260 that fluidly connects the controller 300 to the dressing 200. The dressing base 220 includes flanges 224 that are configured to be positioned generally parallel to the skin of the subject to brace the dressing base 220 against the skin and provide support to the catheter C and the tubing 260. The dressing base 220 additionally may include a saddle 226 that extends away from the flanges 224. The saddle 226 is configured to support and retain the tubing 260 and the catheter C. As shown in FIG. 10, the saddle 226 includes a pair of dressing ports 225 through which tubing 260 is inserted and a central valley 227 formed therebetween that is configured to retain and support the catheter C. According to some inventive embodiments, the dressing ports 225 each include a groove 229 configured to receive a pin 262 that extends from the tubing 260. The interaction of the pin 262 and groove 229 lock the tubing 260 into the dressing ports 225. According to still other inventive embodiments, the valley 227 includes a plurality of ridges 228 that are configured to provide strain relief to the catheter C. These ridges 228 are sized and shaped to engage with corresponding ridges provided on the cover 230.

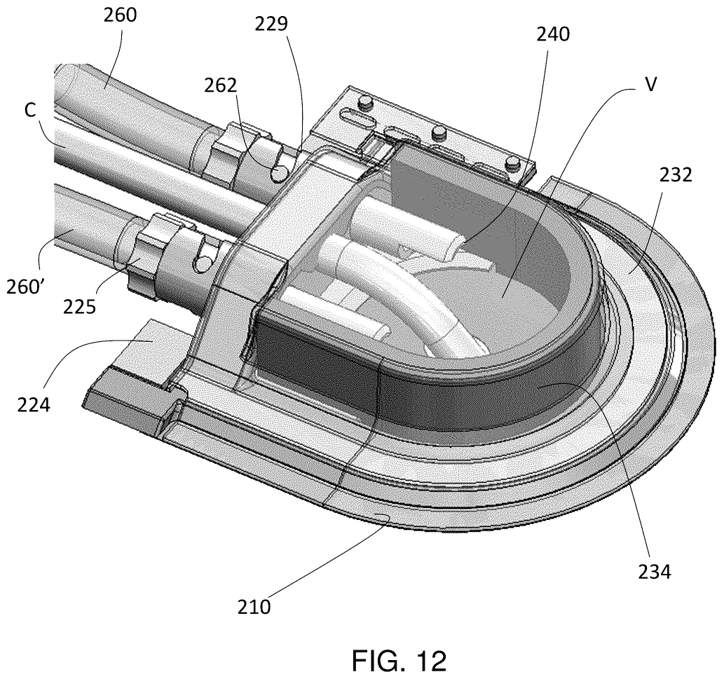

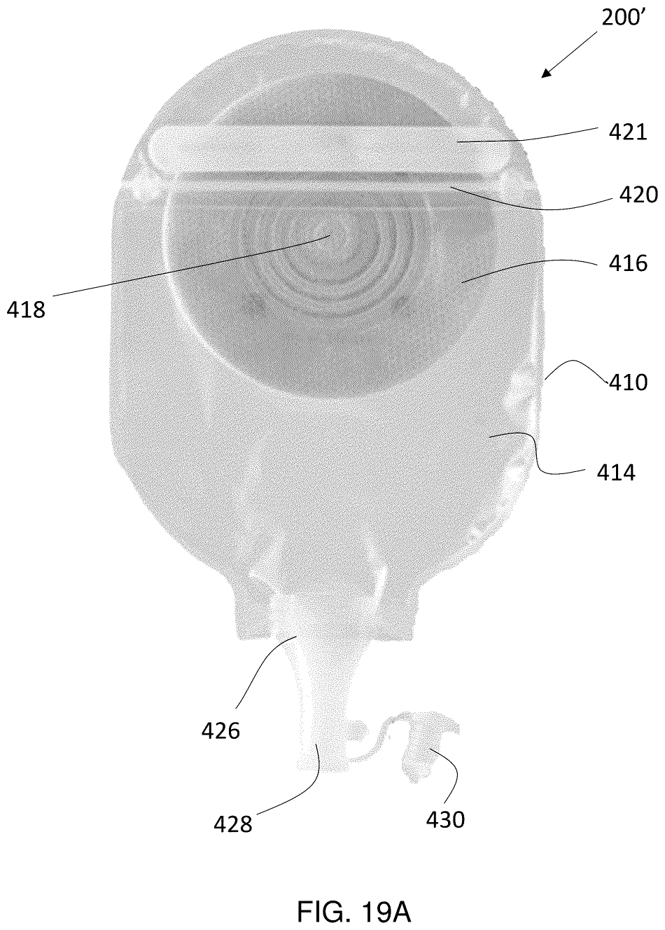

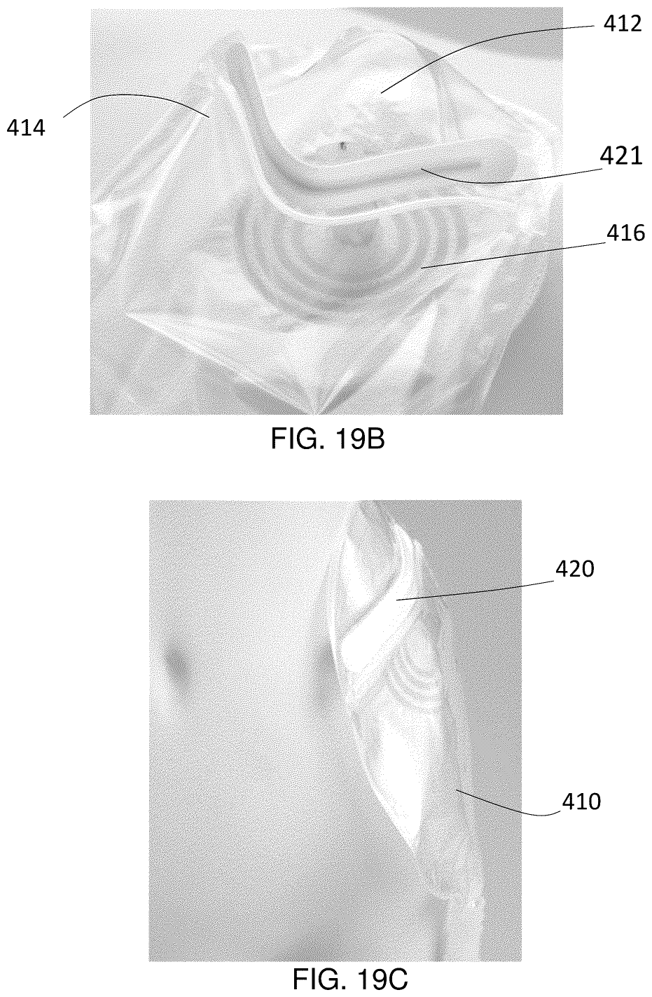

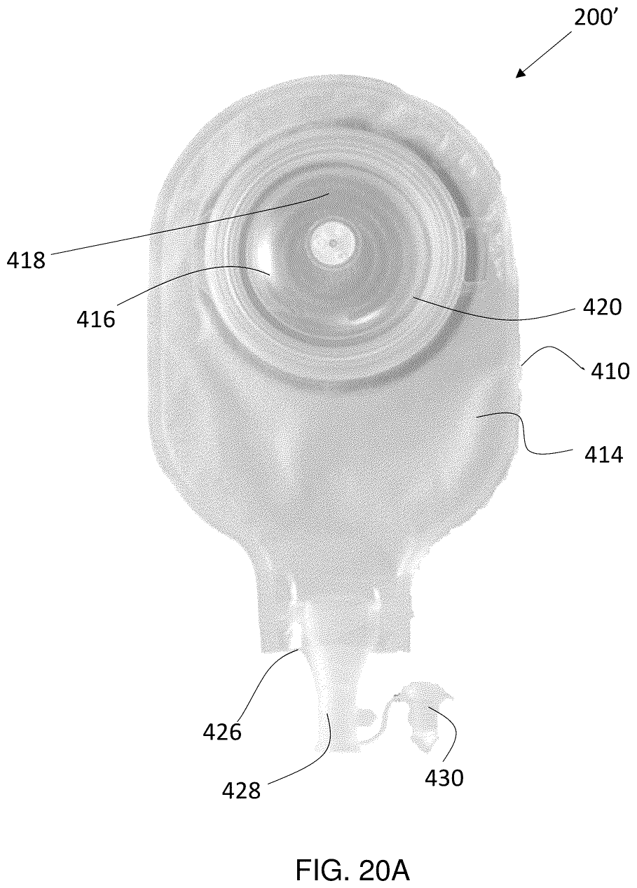

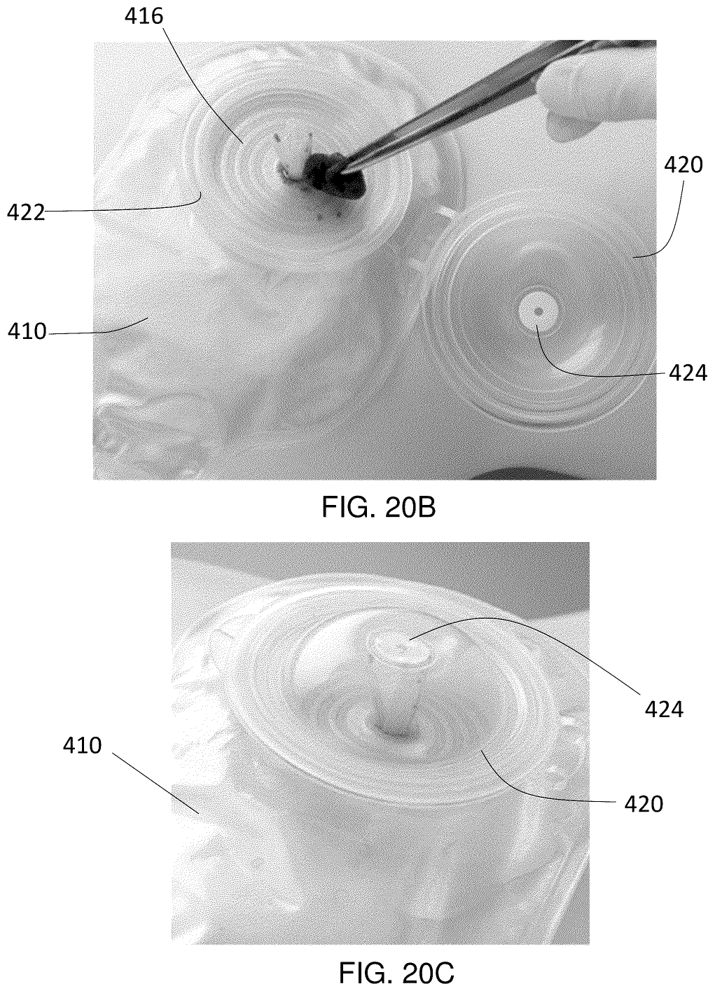

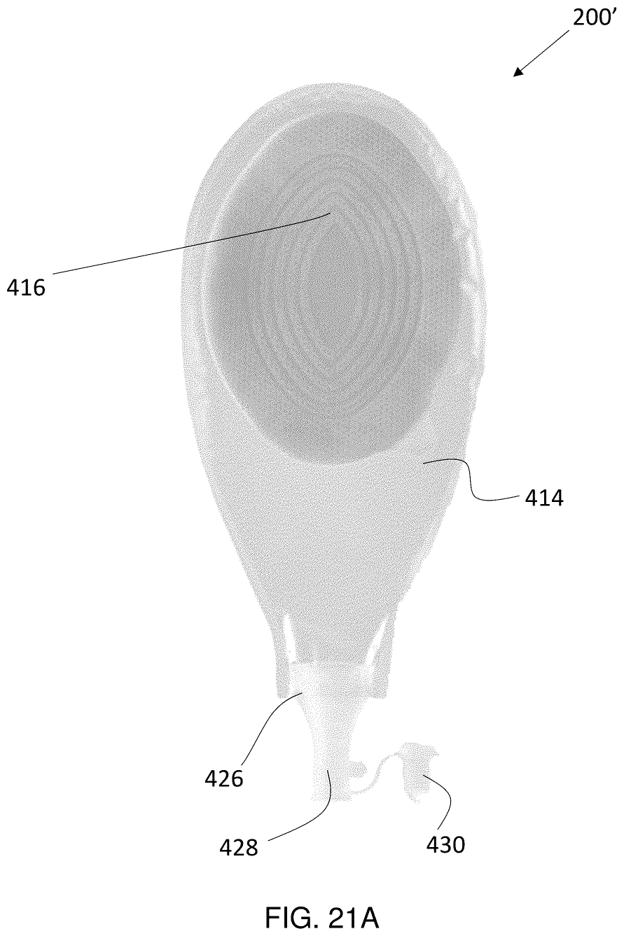

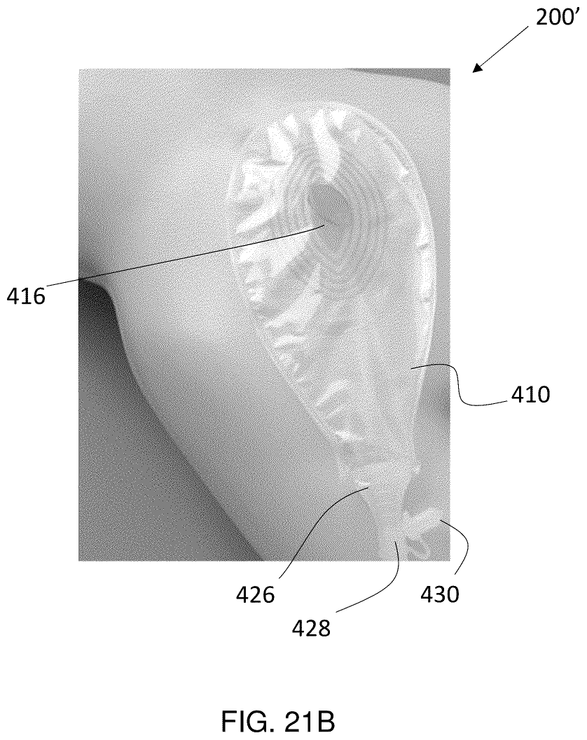

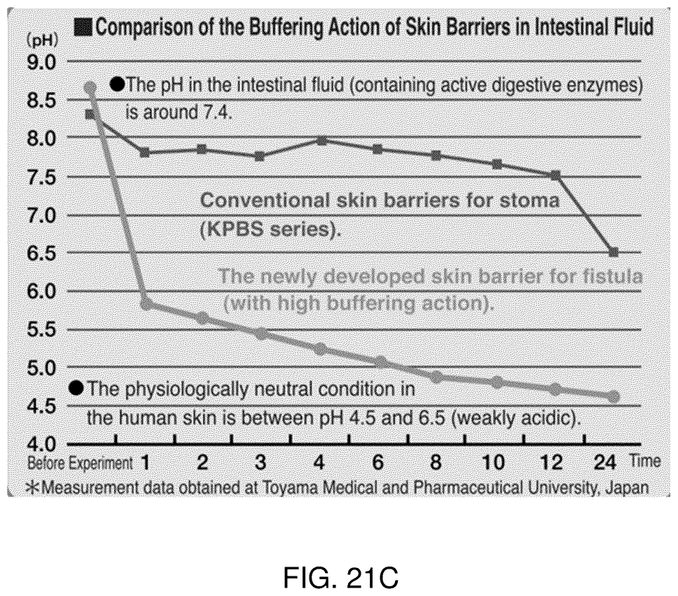

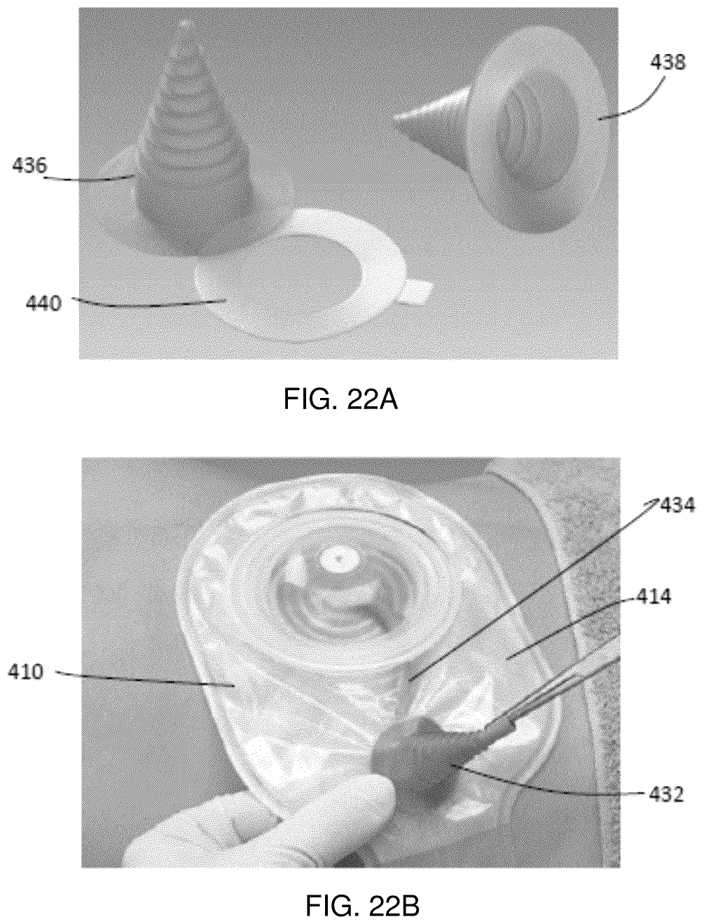

According to some inventive embodiments of the invention, the cover 230 is configured to be attached to the dressing base 220 and to seal to the skin interface 210. According to still other inventive embodiments, the cover 230 is formed of a polyurethane or PVC or other flexible housing. According to other inventive embodiments the cover 230 is pivotably attached to the dressing base 220. The cover 230 includes a flange 232, a wall 234 extending therefrom, and a transparent window 236. According to still other inventive embodiments, the wall 234 includes a saddle receiving portion 235 that corresponds in size and shape to the saddle 226 of the dressing base 220. The saddle receiving portion 235 includes a flexible seal 231 and a plurality of ridges 233 that correspond to the ridges 228 of the dressing base 220. The saddle receiving portion 235 engages with the saddle 226 of the dressing base 220 to lock the cover 230 in a closed position, as shown in FIG. 12, and to further provide support and strain relief to the catheter C and the tubing 260, 260′. According to still other inventive embodiments, the flange 232 is a smooth surface that is configured to engage with the release liner 214. According to embodiments, the flange 232 is provided with a gasket or adhesive seal material 237 to ensure a sealed interface between the cover 230 and the skin interface 210. According to other inventive embodiments, the adhesive material 237 is a hook and loop fastener to ensure that the cover 230 remains in sealed contact with the skin interface 210. According to still other inventive embodiments, the wall 234, the transparent window 236, and the saddle receiving portion 235 define a volume V internal to the cover 230. The volume V is configured to cover the skin portion and the catheter C extending therefrom with the transparent window 236 providing visual access thereto. The transparent window 236 thereby providing a window for medical personnel to visually observe the condition for excessive exudate and skin distortion without needing to remove the dressing. According to still other inventive embodiments, within the volume V the wall 234 is lined with an open cell foam 239. According to other inventive embodiments, the open cell foam 239 polyurethane foam positioned within the cover 230. The open cells of the foam enable equal distribution of the negative pressure across the surface of the wound. In other embodiments of the invention, the atmospheric-facing lid and rigid walls of the visualization chamber are not present and, rather, the atmosphere-facing surface of the dressing, together with the sidewalls of the visualization chamber, are fashioned so as to collapse, under the influence of the applied negative-pressure therapy, to approximately the level of the epidermal-facing aspect of the dressing. This apposition, of the atmospheric-facing surface of the dressing with the epidermis-facing surface of the dressing, addresses the concern that applied negative-pressure wound therapy can result in blistering and de-epithelialization of the skin. Similar to prior-art dressings, as depicted in FIGS. 19A, 19B, 19C, 20A, 20B, 20C, a means is provided to open the visualization chamber to inspect, and care for, the skin and wound adjacent to the medical appliance without disrupting the adhesion of the dressing to the skin; this will reduce the need for frequent dressing changes with their attendant skin irritation. Similar to prior-art dressings as depicted in FIGS. 21A, 21B, 22A, 22B, the dressing chamber can have size, geometry and appliance securement features which will allow for kink-free transit of the medical appliance from the skin-exit site to the appliance exit site of the dressing. Said referenced size, geometry, and appliance securement features can provide for enhanced strain relief of the medical appliance in the vicinity of its skin exit site as well as providing for reliability and ease of vacuum sealing of the dressing to the medical appliance at the appliance exit site of the dressing.

At least one tube 260 that is held in a dressing port 225 of the saddle 226 of the dressing base 220 is a vacuum line that is contented to an exudate collection canister 400 and a vacuum pump 401. According to embodiments, the tube 260 in which exudate is pumped includes a collection port from which exudate may be sampled. The collection canister 400 collects and maintains wound exudate for biohazard disposal. According to embodiments, the canister 500 includes a gel moisture material therein to prevent exudate from getting into the pump 401 and thereby protect the pump 401 from damage by any moisture drawn into the canister 400. The vacuum pump 401 is configured to draw a vacuum within the volume V when the cover 230 is in the closed positioned and sealed to the release liner 214. This tube 260 is also configured to remove exudate from the site for collection within the canister 400. According to embodiments, the pump 400 and/or the collection canister 400 are provided within the same housing as the controller 300. According to embodiments, the canister 400 is disposable and provided within the housing of the controller 300. The canister 400 is configured to be accessed by a user so that the canister may be emptied or changed should the liquid level become full. The canister 400 snaps in securely to the controller 300. According to embodiments, the canister 400 is configured with a valve that prevents spills of the exudate contained therein. According to embodiments, the canister 400 is clear so that a user may visualize the contents therein. According to embodiments, the canister 400 has a stabilizer agent present therein, such as isolyser material to solidify the contents thereof to reduce concerns associated with biohazard material.

A second tube 260′ that is held in a dressing port 225 of the saddle 226 of the dressing base 220 is an active vent tube that connects the volume V within the cover 230 to a controller 300. The active vent tubing 260′ may include a set of wires connected to a pressure sensor, a humidity sensor, a temperature sensor, a volatile chemical sensor, a water-soluble chemical sensor and/or a physiologic sensor 240 that is configured to measure physiologically significant parameters near the site within the dressing 200. According to some inventive embodiments, the sensor 240 is a TruStability Board Mount Pressure Sensor—SSC Series with ±1.6 mbar to ±10 bar (±160 Pa to ±1 MPa; ±0.5 in H2O to ±150 psi) with a digital or analog output. According to other embodiments, the humidity sensor 240 is a Honeywell Humidlcon Digital Humidity/Temperature Sensor 4-pin (HIH6000 series) with ±4.5% RH humidity accuracy, ±0.5° C. temperature accuracy, −40° C. to 100° C. operating temperature range. An internal controller solenoid is integrated to this line to open and close permitting the vacuum within the dressing to be released or cycled with software control. According to some inventive embodiments, an ambient environment sensor 340 is provided on the housing of the controller 300. The ambient sensor 340 is configured to measure the pressure, humidity, temperature, volatile chemicals, water-soluble chemicals and/or a physiologic parameters of the environment in which the inventive skin environment management system is present. These measurements of the ambient environment are used to compare with the measurements taken by the sensor 240 within the dressing 200. According to still other embodiments, the relative humidity is then reported to a user view a display on the controller 300.

The orientation of the dressing 200 is selected by the user as to the direction of the tubing 260 to be placed relative to the controller 300 position. The dressing 200 is applied to the skin of the subject with the bandage 212 being placed thereon and the release liner 214 adhered thereto. The catheter C is placed within the securement feature of the dressing such as the valley 227 of the saddle 226. The openable cover of the dressing, such as depicted as the cover 230 of the dressing 200 is then closed so that the securement features, such as depicted herein by flange 232 contacting the release liner 214 and the saddle receiving portion 235 engages with the saddle 226 of the dressing base 220 to lock the cover 230 in a closed position, as shown in FIG. 12, and to further provide support and strain relief to the catheter C and the tubing 260. The tubing 260, 260′ are then attached to from the dressing 200 to the controller 300 and the collection canister 400, respectively at points 310, 310′.

According to embodiments, the controller 300 includes a battery powered controller with innovations that include: (a) reusable, quiet controller for multiple days of continuous use (A) that delivers controlled vacuum to the dressing; (b) integrated orifice flow restrictor and filter for air exchange (D & inside controller housing); (c) disposable exudate collection canister with filters to protect the controller from contamination (B); (d) optional system controls with a display to permit multiple modes of operation including intermittent, continuous and smart. The smart mode maintains optimum vacuum level based upon relative humidity and temperature sensors that permit cycling the vacuum and introduces moisture and/or anti-infection treatments to the skin environment.

According to some inventive embodiments, the controller 300 is configured to be attached to a belt or strap that a subject fitted with the inventive SEMS 199 may wear. According to still other inventive embodiments, the controller 300 is configured to be placed at a bedside of a subject and connected to the dressing 200 for control thereof. In specific inventive embodiments, a single-use controller has a microprocessor that receives input from the dressing sensors and compares the signals to the controller sensors to adjust the vacuum levels and/or to turn the vacuum on and off. The system 199 controls a diaphragm pump/motor assembly powered by four (4) AA batteries, a display screen, an interlock for canister engagement, battery pack, physiologic sensor and a controllable-leak flow restrictor. The controller is configured to operate, by way of example, for up to seven days to permit the tissue to heal and engage the catheter C. Alarms/Alerts provide visual and audible alerts to the user on a display and from a speaker, respectively. According to embodiments, the alerts include any of alerting that the canister is not engaged, alerting that the canister is full, alerting that flow of exudate is blocked, alerting that the controller is ON/OFF, alerting that the battery is charging, alerting that the vacuum seal is not adequately effective and alerting that the battery is low.

According to some inventive embodiments, the controller includes a plurality of buttons configured to receive user inputs, a plurality of lights, and a numeric display. The buttons include any of an on/off button, an alarm settings menu button, and a button from controlling the humidity/temperature sensor. Alternatively, the system can be controlled remotely, via radiofrequency link, infra-red link, ultrasonic link, and/or other data link, by using a remote smartphone, handset controller, or other remote control input/output device.

According to some inventive embodiments, the controller is programmable. According to still other inventive embodiments, the controller is configured to receive inputs through the local buttons to program to the controller to any of the following features, namely, a system leak time out (seconds), a pressure level setting (mm Hg), a pressure level tolerance (+/−%), Pressure out-of-range (>10% of setpoint—red flashing light & alarm), Vent Interval Program, and Vent Duration (seconds).

According to some inventive embodiments, the controller 300 is a software controlled system that applies negative pressure to the skin portion. The user can select: Continuous or Intermittent/Feedback Pressure Control (FPC) therapy on the therapy unit, depending upon wound type and the needs of each subject. In the FPC mode, two humidity/temperature sensors 240, 340 are utilized (one in the dressing and one in the controller 300) resulting in humidity & temperature differential between the outside environment and the contained volume. Feedback from the humidity sensor can control/adjust the vacuum level based upon dryness (with little exudate from the wound). This can be for example reducing the vacuum level in the dressing to −50 mm Hg compared to normal vacuum level of −125 mm Hg. The Intermittent Control mode cycles the vacuum between and on and an off state, for example of −125 mm Hg for 5-minutes ON and 2-minutes OFF, cycling for 24-hours per day for multiple days. The Continuous Control mode may be −125 mm Hg continuously for multiple days. Alternatively, the vacuum set point can be varied simultaneously at low frequency and high frequency regimes. Alternatively, controls may be provided to the user to adjust the vacuum to a different level. Optionally, additional sensor inputs, derived from sensors embedded within the dressing or positioned in tubing or in the pump/controller, to sample the air returning from the dressing as allowed by the algorithmically-controlled or non-algorithmically controlled leak, can augment the feedback vacuum control algorithm

According to some inventive embodiments, the foam within the dressing provides a tactile/visual indication of vacuum ON/OFF and cycling conditions. The foam also serves to absorb exudate until evacuated to the collection canister or dissectated by the changeover of the atmosphere within the dressing afforded by the combined action of the controlled leak and the vacuum pump. The controller may be a direct current (DC) powered pressure regulator that delivers negative pressure to the dressing 200 ranging from −30 mm Hg to −125 mm Hg or zero mm Hg to −125 mm Hg. The set point (threshold) for the optimal percent humidity is a critical consideration when developing the humidity/temperature algorithm for automated control of the inventive SEMS. In a specific inventive embodiment, the rationale for selecting 92% humidity is based on the need to maintain a moist environment in the volume of the dressing while keeping the environment less than 100% humid. The percent humidity detected by the humidity sensor can be adjusted to any threshold over the entire range from 0% to 100%. According to some inventive embodiments, the SEMS includes a tube configured to introduce moisture into the dressing in the event the humidity sensors indicate that the humidity within the dressing falls below the set point (threshold) for the optimal percent humidity.

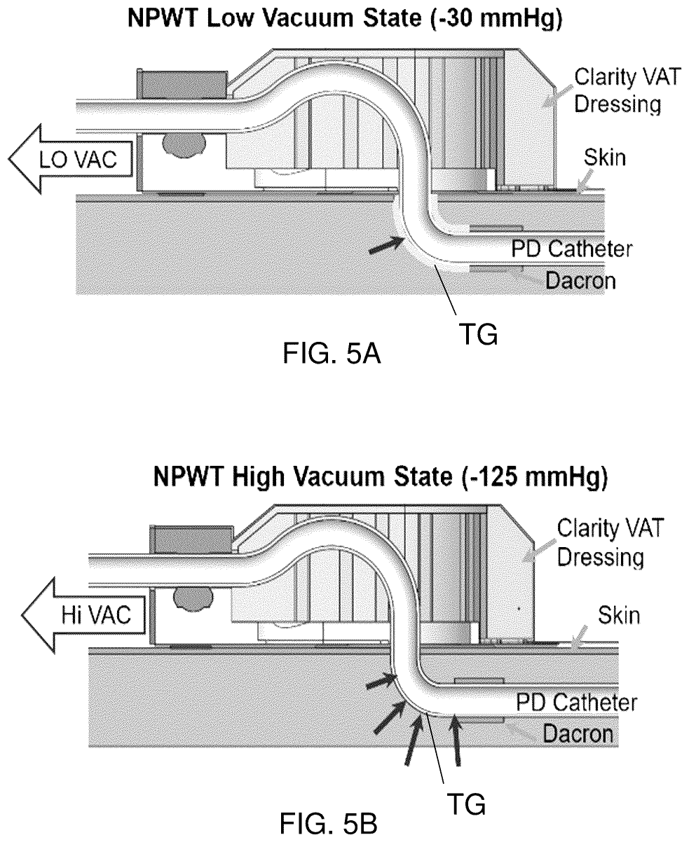

In some embodiments of the inventive SEMS, controlled negative pressure wound therapy (NPWT) is applied to aspirate exudate and bioburden carrying microorganisms out of the volume or in particular, a peri-catheter region while protecting the surrounding skin from harsh vacuum exposure. During a NPWT low vacuum state as shown in FIG. 5A, exudate accumulates within tunnel gap (TG). During a NPWT high vacuum state as shown in FIG. 5B, bioburden/exudate is expressed (as shown by multiple arrows) from within the diminished tunnel gap TG and the adjacent subcutaneous tissue is approximated to the contour of the DACRON® cuff and outer wall of PD catheter. In specific inventive embodiments the duration of use for each dressing will be up to seven days. Embodiments of the SEMS are designed to promote rapid approximation of the tissue to the catheter by providing controlled vacuum up to approximately 125 mm Hg and both mechanical and vacuum-based stabilization of the composite construct of the PD catheter/adjacent tissues/SEMS dressing during the healing process.

A central element of the inventive SEMS is the vacuum assist technology (VAT) dressing that provides the benefits of NPWT to bear on skin environment management around the exit site of a catheter C. The extensive investigations of the mechanism of action of NPWT by Orgill has led to the proposed four basic mechanisms of action: 1) macro-deformation or healing shrinkage; 2) microdeformation or micromechanical cellular changes at the wound-interface surface; 3) removal of fluids; and 4) maintenance of a moist volume. Orgill found that granulation tissue formation is affected by the time and frequency of application of vacuum to the wound environment.

Embodiments of the inventive SEMS are designed to: (1) refresh the volume with filtered air; (2) permit direct visualization to monitor healing and early signs of infection; (3) monitor relative humidity and temperature within the volume, (4) remove exudate/bioburden; (5) control and modulate vacuum relative to the pressure, humidity, and temperature with feedback control at the medical appliance exit site; and (6) treat the volume with anti-infection treatments when signs of infection have been detected.

Embodiments of the inventive SEMS have integrated pressure, relative humidity, and temperature sensors 240 to measure water vapor production adjacent to the medical appliance exit site. Embodiments of the inventive SEMS include integrated air sensors that monitor the air within the volume for chemicals associated with clinically significant microbiologic growth or infections. In such embodiments, the inventive SEMS may additionally include an introducer configured to introduce anti-infection treatments, such as antibiotics, into the volume when the integrated air sensors detect chemicals associated with an infection in the air sampled from the volume. This allows the detected infection to be treated promptly. According to embodiments, tubing 260′ includes slow leak valve that enables air exchange within the dressing 200. A series of algorithms enable vacuum control of the skin environment based upon water vapor production metrics, as well as avoidance of unintended vacuum leaks throughout the dressing. Controllable flow restrictors are optimized for air exchange rates, and the algorithms compare humidity and temperature in the volume to the conditions outside the volume to control the ON/OFF vacuum cycle to remove moisture and draw trapped fluid from around the volume and provide a fully vacuum sealed system. Visualization of the volume is provided to a user through a window positioned above the medical appliance exit access site.

Embodiments of the invention monitor and dynamically control levels of humidity and pressure to optimize skin healing about an implanted device or a wound itself underling an inventive wound dressing. Embodiments of the method and system for actively assessing skin closure are appreciated to be amenable to be incorporated into the design of percutaneous skin access devices (PAD), bone anchors, or a wound dressing or bandage, or any of the other aforementioned medical appliances. The pressure and humidity sensor provide active feedback for making changes to the ecology of the volume overlying the skin portion. In specific inventive embodiments a filter, which illustratively includes a submicron filter, is used to aerate the volume while also preventing pathogens in the ambient air from reaching the wound.

In certain embodiments of the present invention, an assessment of hermaticity may be determined with measurements of humidity in the vacuum line to an inventive dressing. The humidity readings may be taken with impedance humidity sensors. In still other embodiments, local tissue oxygenation in the immediate vicinity of the dressing or PAD or other physiologically important measurements may be used to assess and/or optimize healing.

In certain embodiments of the present invention, an assessment of air quality may be determined with measurements of chemical sniffing sensors in the vacuum line to a dressing or PAD. These sensors are capable to sniffing the air, emitted from the wound, in the vacuum line for chemicals associated with infection. The air quality readings may be taken with air sniffing sensors. Such an air sniffing/air quality sensor illustratively tests for oxygen, or sulfur; exudate biochemical such as electrolytes such as sodium, potassium, or chloride; small molecules such as urea, creatinine, fibrinogen, matrix metalloproteinases (MMPs); large molecules such as tumor necrosis factor (TNFα) and C-reactive protein (CRP); and combinations thereof. Artificial intelligence type algorithms may be applied to the physiologic parameters and the output of the chemical sniffing sensors to improve the detection sensitivity of the infection-detection algorithms. Provisions, using prior-art methods, are made to share data and apply such prior-art data-mining techniques to data amalgamated from one or more patients.

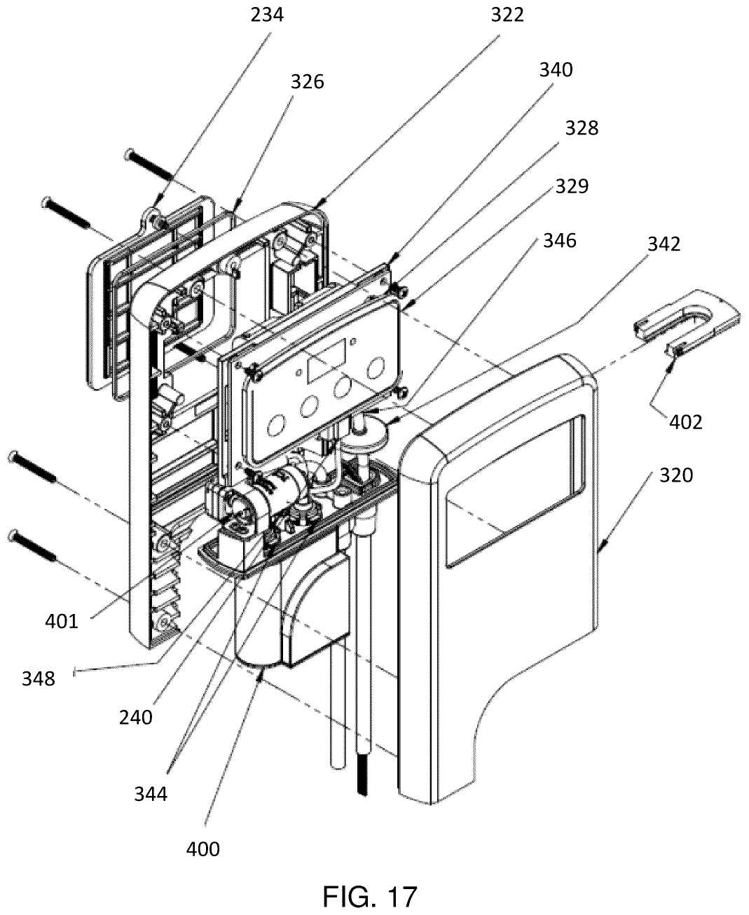

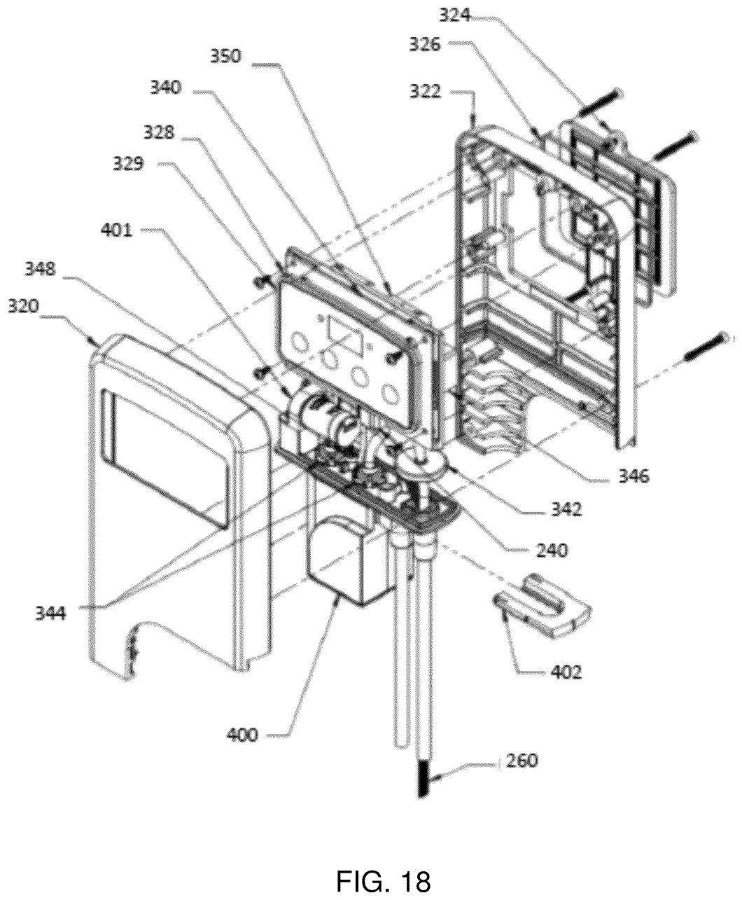

According to some embodiments of the invention, the controller 300 includes a second pump within the housing of the controller 300 that is configured to pump air into the volume V, which according to embodiments is based on the measurements from the chemical sniffing sensors. As shown in FIG. 17, embodiments of the controller 300 include an integrated air flow restrictor. The flow restrictor is utilized to exchange the environmental air within the dressing 200. Target exchange is one exchange per hour in some embodiments. According to embodiments, the volume within the dressing 200 is 0.125 L and the flow restrictor then provides approximately 2 ml/minute±1 ml/minute of air flow into the dressing. At −125 mmHg pressure within the dressing the approximate restrictor orifice is 17 microns when a through hole leak is utilized. Additionally, a sterile filter is provided in some inventive embodiments, which according to embodiments is a 0.22 micron sterile filter.

The hermeticity, temperature, and/or air quality measurement parameters are readily communicated by wired or wireless connection to a computing or communication device for immediate or remote monitoring. Known and future wireless standards and protocols such as, but not limited to, Bluetooth, Zigbee, WiFi, and others may be used to transmit hermeticity, temperature, and/or air quality measurements. Remote monitoring may be facilitated via an Internet or cellular network enabled device in communication with the output of a hermeticity measurement device or sensor. The hermeticity, temperature, physiologic, and/or air quality measurement devices or sensors may require an external power source such as a battery or may be passive elements such as radio frequency identification elements (RFID), which obviate the need for an electrical power source to be directly incorporated into the PAD or dressing. A passive RFID element retransmits a signal using the energy of an incoming interrogation signal, where in embodiments of the inventive hermaticity sensor, temperature sensor, physiologic, and/or air quality sensor the transmitted signal will vary in frequency or phase with the respective measurement. In certain embodiments, battery power used to supply the vacuum source of the dressing may also be utilized to supply power to the one or more hermeticity, temperature, physiologic, and/or air quality sensors.

The hermeticity, temperature, physiologic, and/or air quality sensors measurement information is readily employed for local closed-loop control of the vacuum supply to the dressing, and to alert the patient and/or care giver with regards to progress or problems with the dressing. Additionally, the hermeticity, temperature, physiologic, and/or air quality information may be transmitted wirelessly to medical personnel to allow for remote monitoring of the healing wound. For example, as impedance or humidity in a vacuum line stabilizes, medical personnel may be notified that the wound has healed. Alternatively, if the impedance or humidity deviated from expected values or if an air quality sensor detects a chemical commonly associated with an infection, medical personnel could be notified that there may be an infection or a mechanical disruption to the wound; alarms could also be set to notify the subject. In an embodiment, the vacuum supplied to the dressing could automatically be increased or decreased based on the healing, moisture could automatically be introduced to the volume, and/or an anti-infection treatment could automatically be supplied to the volume.

In specific inventive embodiments, integrated multi-lumen tubing as disclosed in US Patent Publication No. US2020/0289810 is used for delivering a vacuum. Integrated multi-lumen tubing provides a combination of intravenous (IV) infusion lines, vacuum lines, and in some instances monitoring lines for attachment to a percutaneous access device or long-term implant. The integration of the intravenous infusion lines, vacuum lines, and monitoring lines that connect to the dressing and other inserted instruments organizes the myriad of intravenous infusion lines, vacuum lines, and monitoring lines that connect to the dressing or PAD and other inserted instruments that tend to get tangled, interfere with subject comfort and movement, and are potentially difficult for health care workers to change and maintain. Furthermore, by using the lines associated with the IV already present in a hospital or medical facility allows for use of the existing vacuum source used in the facility.

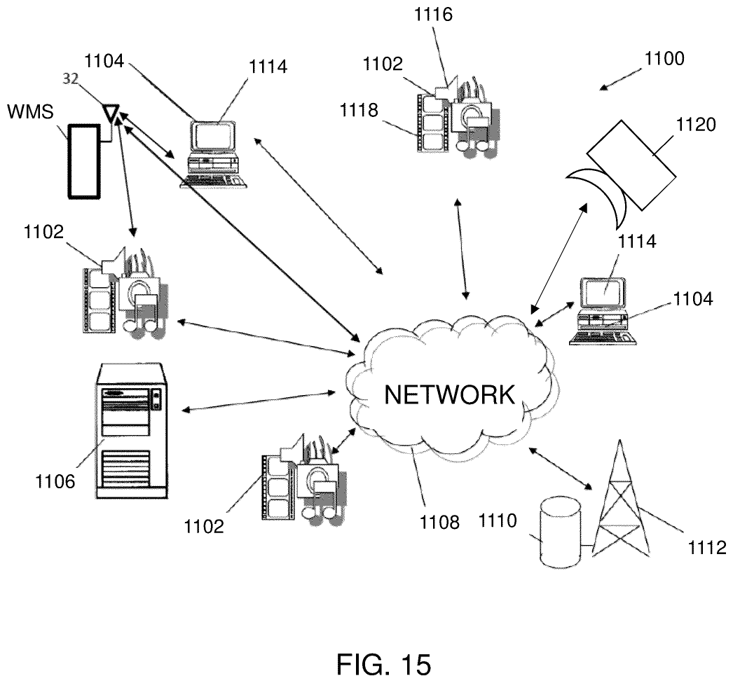

FIG. 15 is a schematic diagram illustrating an overall view of communication devices, computing devices, and mediums for implementing the skin environment monitoring system platform according to embodiments of the invention.

The system 1100 includes multimedia devices 1102 and desktop computer devices 1104 configured with display capabilities 1114 and processors for executing instructions and commands. The multimedia devices 1102 are optionally mobile communication and entertainment devices, such as cellular phones and mobile computing devices that in certain embodiments are wirelessly connected to a network 1108. The multimedia devices 1102 typically have video displays 1118 and audio outputs 1116. The multimedia devices 1102 and desktop computer devices 1104 are optionally configured with internal storage, software, and a graphical user interface (GUI) for carrying out elements of the skin environment monitoring system platform according to embodiments of the invention. The network 1108 is optionally any type of known network including a fixed wire line network, cable and fiber optics, over the air broadcasts, satellite 1120, local area network (LAN), wide area network (WAN), global network (e.g., Internet), intranet, etc. with data/Internet and remote storage capabilities as represented by server 1106. Communication aspects of the network are represented by cellular base station 1110 and antenna 1112. In a preferred embodiment, the network 1108 is a LAN and each remote device 1102 and desktop device 1104 executes a user interface application (e.g., Web browser) to contact the server system 1106 through the network 1108. Alternatively, the remote devices 1102 and 1104 may be implemented using a device programmed primarily for accessing network 108 such as a remote client. Hermeticity/temperature/pressure/physiologic, and air quality sensors in the skin environment monitoring system may communicate directly or via the controller 300 with remote devices 1102 and 1104 via near field communication standards such as Bluetooth or Zigbee, or alternatively via network 1108. In addition, the dressing may be combined with ultrasound to promote collagen deposition and improve wound healing (https://www.physio-pedia.com/Ultrasound_in_Wound_Healing), or augmented by an oscillating vacuum pressure set point.

The software for the skin environment monitoring system platform, of certain inventive embodiments, is resident in the controller 300, on multimedia devices 1102, desktop or laptop computers 1104, or stored within the server 1106 or cellular base station 1110 for download to an end user. Server 1106 may implement a cloud-based service for implementing embodiments of the platform with a multi-tenant database for storage of separate client data.

As shown in greater detail in FIGS. 19A-23B, in which like reference numerals have the same meaning ascribed thereto with respect to previously detailed drawings, further embodiments of a dressing 200′ are shown. Advantageously, in the embodiments, the dressing 200′ is flat, flexible, and compact, which increases comfort and wearability for the patient. In such embodiments, the wound dressing 200′ includes a pouch 410 that is formed of two layers 412, 414 of flexible sheet material, such as polyethylene. The two sheets 412, 414 are sealed together along their edges to define a sealed chamber therein. According to embodiments, the pouch 410 is available in a variety of sizes so that the sealed chamber is of different volumes. The dressing 200′ additionally includes a skin barrier 416 fixed to a sheet 412 of the pouch 410 and that is configured to adhere the skin barrier 416 to the skin of a patient around a wound site. The pouch 410 additionally includes an opening 418, in the same sheet 412 that the skin barrier 416 is attached to, positioned such that the skin barrier 416 aligns with the opening 418 so that the opening 418 and the wound site may be aligned, when the skin barrier 416 seals with the skin of the patient. According to embodiments, the pouch 410 is clear so that the wound site is visible through the sheets 412, 414.

According to embodiments, the dressing 200′ additionally includes a sealable access opening 420 through the second sheet 414 of the pouch 410 so that the interior of the sealed chamber within the pouch is accessible while the pouch 410 remains in adhesion and sealed to the skin of the patient around the wound site. As shown in FIGS. 19A-19C, the sealable access opening 420 is a zip seal that is configured to be opened and closed to allow and restrict access of the chamber within the pouch 410. According to such embodiments, the zipper is outfitted with a flexstick 421 that is configured to resiliently hold the zipper open so that the access opening 420 is easily accessible. According to further embodiments, such as that shown in FIGS. 20A-20C, the sealable access opening 420 is a dome-shaped cap that is configured to open and close in a snap engagement with a sealing ring 422 attached to the second sheet 414 to allow and restrict access of the chamber within the pouch 410. According to embodiments, the dome-shaped cap is equipped with a back flow valve 424. According to some embodiments, the dome-shaped cap is connected to the pouch 410 to prevent the dome-shaped cap from being lost. Additionally, the dome-shaped cape protects the wound site from external pressure. Alternatively, that portion of the resealable cap delimited by the resealable rim remains sufficiently mechanically compliant to collapse, under the influence of the applied vacuum, against the skin-facing layer of the dressing and the medical appliance so as to minimize the risk of bariatric damage of that segment of skin exposed to vacuum conditions (i.e. the epidermis under the dressing is adequately spared exposure to conditions which could lead to vacuum-induced de-epithelization or blister formation. The only region of skin exposed to full vacuum conditions is that narrow annulus of tissue immediately adjacent to the skin entry site of the medical appliance)

According to some embodiments, the skin barrier 416 includes concentric circle seals that fix the skin barrier 416 to the first sheet 412 of the pouch 410. These seals further act to create protective sealed layers when the skin barrier is attached to the skin of a patient. In order to attach to the skin of a patient, the skin barrier 416 includes an adhesive on the side opposite the pouch 410. The adhesive is like that described above and is configured to seal the skin barrier 416 to the skin of a patient such that it strongly adheres to the skin yet remains removable with limited residual adhesive left on the skin upon the removal of the skin barrier 416 from the skin. According to embodiments, the skin barrier 416 is a soft, flexible material. According to some embodiments, the skin barrier 416 is configured to engage in pH buffering, which may be particularly useful in some treatment locations such as when the dressing 200′ is used on a fistula. According to such embodiments, the pH of the intestinal fluid containing active digestive enzymes rapidly decreases from 8.7 to 5.8, as shown in the graph of FIG. 21C, adjusting to the skin physiologically neutral condition in the human skin. Thus, the skin irritation commonly caused by such drainage is prevented due to the high pH buffering action of the skin barrier.

According to some embodiments, the dressing 200′ additionally includes an outlet 426 that is formed in at least one of the sheets 412, 414 of the pouch 410 and that is configured to fluidly connect the internal chamber of the pouch with the environment external to the pouch 410. The outlet 426 allows wound fluids that may have connected within the pouch 410 to be drained from the dressing 200′. According to embodiments, the outlet 426 includes a nozzle 428 and a cap 430 so that the outlet 426 may be closed to maintain a sealed environment within the chamber within the pouch 410. According to embodiments, the cap 30 is configured to threadably engage with the nozzle 428 or engage by way of a friction fit. According to embodiments, the cap 430 is linked to the nozzle 428 to prevent the cap 430 from being lost.

According to some embodiments, as shown in FIGS. 22A and 22B, the dressing 200′ additionally includes a connection fixture 432 attached to at least one of the sheets 412, 414 of the pouch 410 at a through hole therein. According to embodiments, the connection fixture 432 is attached to the pouch 410 by a strong adhesive. The connection fixture 432 is configured to allow a medical appliance such as a catheter or tube to connect to the dressing 200′ According to embodiments, the connection fixture 432 additionally includes a tube 434 that extends from the connection fixture 432 at the through hole to the wound site with the tube 434 positioned within the chamber of the pouch 410. According to some embodiments, the connection fixture is a cone connect device that includes several concentric circles as connection points so that medical appliances of various sizes from 6-40 Fr may easily be fitted to the connection fixture 432 without needing to swap the connection fixture 432 for a different size. According to embodiments, the connection fixture 432 includes three parts, namely a first stepped cone 436, a second stepped cone 438 and a gasket 440. According to such embodiments, gasket 440 is positioned onto the first stepped cone 436, which is then inserted through a through hole in the second sheet 414 of the pouch 410 from the inside of the chamber to the outside of the chamber of the pouch 410. The second stepped cone 438 is then nested onto the first stepped cone 436 as it projects out from the pouch 410. An adhesive may be used to hold the components to the pouch 410. For example, the gasket 440 may include an adhesive thereon for applying the stepped cones to the pouch 410.

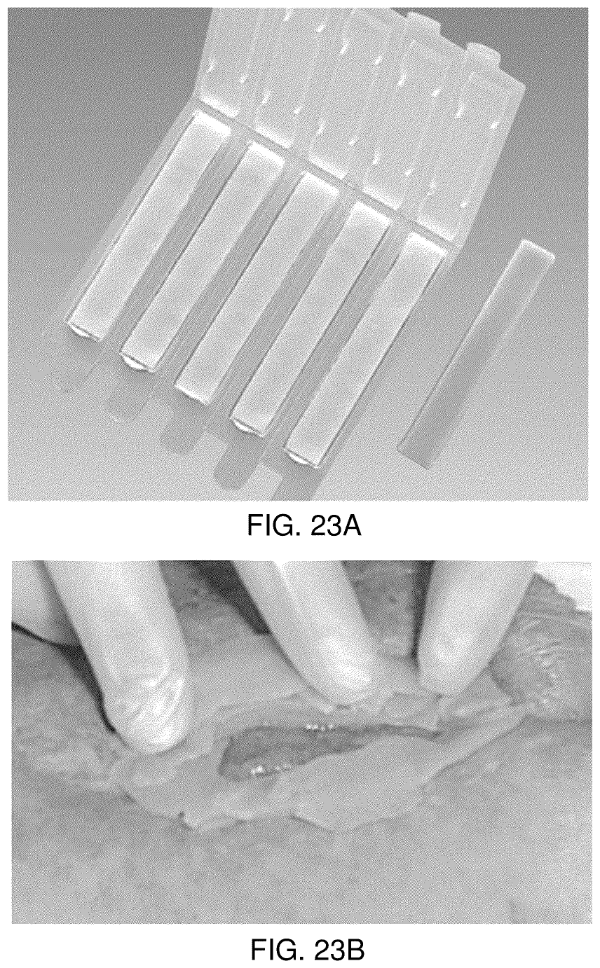

According to some embodiments, the dressing 200′ is configured to be used with a PROCARE soft wafer stick skin barrier, as shown in FIGS. 23A-23B, which according to some embodiments is formed of Karaya Gum and Cirtus Pectin. This flexible, easy to process skin barrier snugly fits an uneven skin surface with wrinkles and hollows to achieve close contact with the pouch 410.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A skin environment management system for controlling environmental conditions on a portion of skin of a subject comprising:
a dressing comprising a skin interface, a dressing base attached to the skin interface, and a cover reversibly-openable and connected to the dressing base to together define a volume, the cover sealable to the skin interface, the dressing configured to be positioned over the portion of the skin, the dressing defining an environment in the volume surrounding the portion of the skin;
a pump in fluid communication with the environment by a first tube, the pump configured to apply a reduced pressure vacuum to the environment; and
a controller configured to dynamically control operation of the pump.

2. The skin environment management system of claim 1 wherein the skin interface includes a through hole through which at least a portion of the implantable medical device is configured to pass, a bandage portion configured to be in direct contact with the skin, a release portion configured to seal to a subject's skin, or a combination thereof.

3. The skin environment management system of claim 2 wherein the bandage portion is present and free of an adhesive.

4. The skin environment management system of claim 2 wherein the release portion is present and includes an adhesive on a first side thereof.

5. The skin environment management system of claim 1 wherein the dressing base includes a plurality of flanges configured to be placed upon the subject's skin, a saddle or securement feature configured to support and retain the implantable medical device and the first tube, or a combination thereof.

6. The skin environment management system of claim 5 wherein the saddle or securement feature defines at least one dressing port to which the first tube connects or a valley that is configured to receive and support the implantable medical device and reduce strain thereon.

7. The skin environment management system of claim 6 wherein the at least one dressing port includes a groove configured to receive and retain a pin provided on a first end of the first tube.

8. The skin environment management system of claim 1 wherein the cover includes a flange sealable to the skin interface, a wall extending from the flange, and a transparent, and optionally reversibly-openable window.

9. The skin environment management system of claim 8 wherein the wall includes a saddle receiving portion or securement feature that is configured to engage with the dressing base, foam on the wall within the volume, or a combination thereof.

10. The skin environment management system of claim 1 further comprising a fluid collection canister in fluid communication with the volume by the first tube.

11. The skin environment management system of claim 1 further comprising at least one of a humidity sensor, a pressure sensor, a temperature sensor, a physiologic sensor, and an air quality sensor integrated into the dressing or the controller to provide physiologic parameters that correlate to any of a degree of healing, functional integrity of the skin environment management system, and an infection state within the environment.

12. The skin environment management system of claim 1 further comprising a second tube connecting the volume to the controller and the second tube is an active vent, houses a plurality of wires that electrically connect the controller to at least one sensor positioned within the volume, or a combination thereof.

13. A dressing for use with a skin environment management system, the dressing comprising:
a pouch formed of a first sheet and a second sheet layered together and sealed along an outer edge to define a chamber therein;
a skin barrier attached to the first sheet at a through hole in the first sheet; and
a sealable outlet configured to fluidly connect the chamber of the pouch with an environment external to the chamber of the pouch.

14. The dressing of claim 13 further comprising a sealable access opening provided in the second sheet of the pouch.

15. The dressing of claim 14 wherein the sealable access opening includes a zipper, a dome-shaped cap, or a combination thereof.

16. The dressing of claim 15 wherein the zipper is present and includes a flexstick configured to hold the access opening in an open position when the zipper is unsealed.

17. The dressing of claim 15 wherein the dome-shaped cap is present and configured to open and close in a snap engagement with a sealing ring provided on the second sheet of the pouch and optionally includes a backflow valve.

18. The dressing of claim 13 further comprising a connection fixture extending from the pouch about a second through hole therein.

19. The dressing of claim 13 wherein the first sheet and the second sheet that form the pouch are transparent.

20. The dressing of claim 13 wherein the skin barrier is configured to adhere to a patient's skin around a wound site, engage in pH buffering, or a combination thereof.
